(19) United States
(12) Patent Application Publication
(10) Pub. No.: US 2015/0197513 A1
(43) Pub. Date: Jul. 16, 2015

(54) ARYL- AND HETEROARYL-SUBSTITUTED BENZENE DERIVATIVES AS MODULATORS OF PI3-KINASE SIGNALLING PATHWAYS

(71) Applicant: Neuropore Therapies, Inc., San Diego, CA (US)

(72) Inventors: Wolfgang Wrasidlo, San Diego, CA (US); Emily M. Stocking, Encinitas, CA (US)

(21) Appl. No.: 14/420,315

(22) PCT Filed: Aug. 8, 2013
(86) PCT No.: PCT/US13/54200

§ 371 Date: Feb. 6, 2015

Related U.S. Application Data
(60) Provisional application No. 61/681,585, filed on Aug. 9, 2012.

(51) Int. Cl. C07D 417/12; C07D 257/06; C07D 277/42; C07D 233/88; C07D 207/34; C07D 285/135; C07D 271/113

(52) U.S. Cl. CPC C07D 417/12; C07D 285/135; C07D 257/06; C07D 271/113; C07D 233/88; C07D 207/34; C07D 277/42

(57) ABSTRACT

The present disclosure relates to certain aryl- or heteroaryl-substituted benzene derivatives, pharmaceutical compositions containing them, and methods of using them, including methods for modulating autophagy or preventing, reversing, slowing or inhibiting the PI3K-AKT-MTOR pathway, and methods of treating diseases that are associated with autophagy or the PI3K-AKT-MTOR pathway.

### CROSS-REFERENCE TO RELATED APPLICATIONS

This application claims priority to U.S. Provisional Patent Application No. 61/681,585, filed Aug. 9, 2012, which is incorporated herein in its entirety.

### TECHNICAL FIELD

The present disclosure relates to aryl- and heteroaryl-substituted benzene compounds, pharmaceutical compositions containing them, and methods of using them, including methods for modulating the PI3K-AKT-MTOR pathway, methods for activating, increasing or stimulating autophagy by preventing, reversing, slowing or inhibiting the PI3K-AKT-MTOR pathway, and methods for treating diseases that are associated with mis-regulation of the PI3K-AKT-MTOR pathway.

### BACKGROUND

Autophagy, a principal mechanism for the clearance of cellular constituents, plays an important role in development, cellular differentiation, homeostasis, and cell survival. Mis-regulation of autophagy has been linked to a number of different neurodegenerative disorders including amyotrophic lateral sclerosis, Alzheimer's disease, Parkinson's disease and Huntington's disease. Therapeutic agents that activate autophagy may be beneficial in the treatment of these neurodegenerative disorders (Martinez-Vicente et al. Nat. Neurosci. 2010, 13(5), 567-576).

The PI3K-AKT-mTOR (PI3 kinase/Akt/mammalian target of rapamycin) pathway regulates the expression of cell survival genes and cell energetics. This pathway is also a key negative regulator of autophagy (Codogno and Meijer, Cell Death Differ. 2005, 12(S2), 1509-1518, Bhaskar, et al. Molecular Neurodegeneration 2009, 4, 14; Cherra and Chu, Future Neurol. 2008, 3(3), 309-323). Thus, inhibition of the PI3K-AKT-mTOR pathway may be an ideal way to up regulate autophagy, promote cell survival, and treat neurodegenerative disorders.

### SUMMARY

There remains a need for compounds that effect autophagy with desirable pharmaceutical properties. Certain aryl- and heteroaryl-substituted benzene compounds have been found to inhibit the PI3K-AKT-MTOR pathway. These compounds inhibit phosphorylation of AKT and mTOR. Consequently, these compounds also increase markers of autophagy and increase cellular clearance of toxic protein aggregates. These compounds may thus have utility in the treatment of neurodegenerative disorders and other disorders associated with the PI3K-AKT-mTOR signaling pathway.

In one aspect, the present disclosure provides a compound of Formula (I):

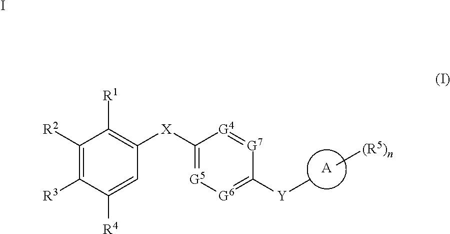

wherein
R¹, R², R³, and R⁴ are each independently hydrogen, hydroxy, halogen, C₁₋₄ alkyl, substituted C₁₋₄ alkyl, C₁₋₄ alkoxy, substituted C₁₋₄ alkoxy, —CN, —COR^{x}, —CO₂R^{x}, —SO₂R^{x}, or —NR^{x}R^{y};
   wherein R^{x} and R^{y} are each independently H or optionally substituted C₁₋₄alkyl, or R^{x} and R^{y} taken together with the nitrogen to which they are attached form an optionally substituted monocyclic heterocycloalkyl ring;
X is absent, or is C₁₋₆ alkylene, wherein one carbon unit of said alkylene is optionally replaced with —O—, —S—, —SO—, —NR^{a}—, —SO₂—, or —CO—;
   wherein R^{a} is hydrogen or C₁₋₄ alkyl;
G⁴, G⁵, G⁶, and G⁷ are each independently CR¹⁰ or N;
   wherein each R¹⁰ is independently hydrogen, hydroxy, halogen, C₁₋₄ alkyl, C₁₋₄ haloalkyl, C₁₋₄ alkoxy, or C₁₋₄ haloalkoxy;
Y is absent, or is C₁₋₆ alkylene, wherein one carbon unit of said alkylene is optionally replaced with —O—, —S—, —NH—, —SO—, —SO₂—, —CO—, —CO₂—, —CONH—, —NHCO—, —NHSO₂—, or —SO₂NH—;
Ring A is a 5-membered heteroaryl ring;
each R⁵ is independently C₁₋₆ alkyl, substituted C₁₋₆ alkyl, C₁₋₆ alkoxy, substituted C₁₋₆ alkoxy, C₃₋₈ cycloalkyl, substituted C₃₋₈ cycloalkyl, C₃₋₈ cycloalkoxy, substituted C₃₋₈ cycloalkoxy, hydroxyl, halogen, —NR^{m}Rⁿ, or cyano;
   wherein R^{m} and Rⁿ are each independently H or C₁₋₄alkyl; and
n is a number from zero to three;
   or a pharmaceutically acceptable salt thereof.

In certain embodiments, the compound of Formula (I) is a compound selected from those species described or exemplified in the detailed description herein.

In a further aspect, the present disclosure provides a pharmaceutical composition comprising at least one compound of Formula (I) or a pharmaceutically acceptable salt thereof. Pharmaceutical compositions according to the embodiments may further comprise a pharmaceutically acceptable excipient. The present disclosure also provides a compound of Formula I or a pharmaceutically acceptable salt thereof for use as a medicament.

In another aspect, the present disclosure provides a method of treating a disease or medical condition associated with autophagy or the PI3K-AKT-mTOR pathway, comprising administering to a subject in need of such treatment an effective amount of at least one compound of Formula (I) or a pharmaceutically acceptable salt thereof. In some embodiments, the disease or medical condition is a neurodegenerative disease or condition. The present disclosure provides use of a compound of Formula (I) in the preparation of a medicament for the treatment of such diseases and medical conditions, and the use of such compounds and salts for treatment of such diseases and medical conditions.

In yet another aspect, the present disclosure provides a method of interfering with the process of autophagy in a cell modulating, activating, increasing or stimulating autophagy in a cell or preventing, reversing, slowing or inhibiting the PI3K-AKT-mTOR pathway, comprising contacting the cell with an effective amount of at least one compound of Formula (I) or a salt thereof, and/or with at least one pharmaceutical composition of the embodiments, wherein the contacting is in vitro, ex vivo, or in vivo.

Additional embodiments, features, and advantages of the invention will be apparent from the following detailed description and through practice of the invention.

### BRIEF DESCRIPTION OF THE DRAWINGS

FIG. 1 is a graph that shows a comparison of the effects of different compounds on inhibition of PI3K-AKT-mTor pathway, where the bars represent relative concentrations (RFU) of pAKT in B103 cells (mean±SEM) as determined with a pAKT specific ELISA assay. LY294002 and rapamycin are reference compounds known to modulate the PI3K-AKT-MTOR pathway.

FIG. 2 is a graph that shows a comparison of the effects of different compounds on inhibition of PI3K-AKT-mTor pathway, where the bars represent relative concentrations (RFU) of pMTOR in B103 cells (mean±SEM) as determined with a pMTOR specific ELISA assay.

FIG. 3 is a graph that shows the number of autophagosomes per cell and is a marker of autophagy, where the bars represent LC3-GFP positive puncta counts per cell (mean±SEM).

FIG. 4 is a graph that shows a comparison of compounds on the clearance of oligomeric Amyloid Beta, where the bars represent the concentration of amyloid beta (Aβ) (mean±SEM).

### TERMS

The following terms have the following meanings unless otherwise indicated. Any undefined terms have their art recognized meanings.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), neopentyl ((CH₃)₃CCH₂—), and n-hexyl (CH₃(CH₂)₅—).

“Alkylene” refers to divalent aliphatic hydrocarbyl groups preferably having from 1 to 6 and more preferably 1 to 3 carbon atoms that are either straight-chained or branched. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Haloalkyl” refers to an alkyl group as described above, wherein one or more hydrogen atoms on the alkyl group have been substituted with a halo group. Examples of such groups include, without limitation, fluoroalkyl groups, such as fluoroethyl, trifluoromethyl, difluoromethyl, trifluoroethyl and the like.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

“Alkoxyamino” refers to the group —NH-alkoxy, wherein alkoxy is defined herein.

“Haloalkoxy” refers to the group alkyl-O— wherein one or more hydrogen atoms on the alkyl group have been substituted with a halo group and include, by way of examples, groups such as trifluoromethoxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—.

“Carboxyl,” “carboxy” or “carboxylate” refers to —CO₂H or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-hetero aryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or “aminoacyl” refers to the group —C(O)NR²¹R²², R²¹ wherein R and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

“Acylamino” refers to the groups —NR²⁰C(O)alkyl, —NR²⁰C(O)substituted alkyl, NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxyl ester, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, sulfonylamino, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

“Cycloalkenyl” refers to non-aromatic cyclic alkyl groups of from 3 to 10 carbon atoms having single or multiple rings and having at least one double bond and preferably from 1 to 2 double bonds.

“Cycloalkoxy” refers to —O-cycloalkyl.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxyl ester, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, sulfonylamino, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

Examples of heteroaryls include, but are not limited to, pyrrole, imidazole, pyrazole, pyridine, pyrazine, pyrimidine, pyridazine, indolizine, isoindole, indole, purine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, piperidine, piperazine, phthalimide, 4,5,6,7-tetrahydrobenzo[b]thiophene, thiazole, thiophene, benzo[b]thiophene, and the like.

“Heterocycle,” “heterocyclic,” “heterocycloalkyl” or “heterocyclyl” refers to a saturated or partially unsaturated group having a single ring or multiple condensed rings, including fused, bridged, or spiro ring systems, and having from 3 to 20 ring atoms, including 1 to 10 hetero atoms. These ring atoms are selected from the group consisting of carbon, nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for N-oxide, —S(O)—, or —SO₂-moieties.

Examples of heterocycles include, but are not limited to, azetidine, dihydroindole, indazole, quinolizine, imidazolidine, imidazoline, piperidine, piperazine, indoline, 1,2,3,4-tetrahydroisoquinoline, thiazolidine, morpholinyl, thiomorpholinyl (also referred to as thiamorpholinyl), 1,1-dioxothiomorpholinyl, piperidinyl, pyrrolidine, tetrahydrofuranyl, and the like.

Where a heteroaryl or heterocyclyl group is “substituted,” unless otherwise constrained by the definition for the heteroaryl or heterocyclic substituent, such heteroaryl or heterocyclic groups can be substituted with 1 to 5, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, azido, cyano, halogen, hydroxyl, oxo, thioketo, carboxyl, carboxyl ester, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, sulfonylamino, —SO-alkyl, —SO— substituted alkyl, —SO-aryl, —SO-heteroaryl, —SO-heterocyclyl, —SO₂-alkyl, —SO₂-substituted alkyl, —SO₂-aryl, —SO₂-heteroaryl, and —SO₂-heterocyclyl.

“Heteroaryloxy” refers to —O-heteroaryl.

“Heterocyclyloxy” refers to the group —O-heterocyclyl.

“Azido” refers to the group —N₃.

“Cyano” or “nitrile” refers to the group —CN.

“Halo” or “halogen” refers to fluoro, chloro, bromo, and iodo.

“Hydroxy” or “hydroxyl” refers to the group —OH.

“Hydroxylamine” refers to the group —NHOH.

“Nitro” refers to the group —NO₂.

“Oxo” refers to the atom (═O).

“Keto” refers to the group (C═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cycloalkenyl, SO₂-aryl, SO₂-substituted aryl, SO₂-heteroaryl, SO₂-substituted heteroaryl, SO₂-heterocyclic, and SO₂-substituted heterocyclic, wherein each alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Poly(alkylene glycol)” refers to straight or branched polyalkylene glycol polymers such as polyethylene glycol, polypropylene glycol, and polybutylene glycol. A polyalkylene glycol subunit is a single polyalkylene glycol unit. For example, an example of a polyethylene glycol subunit would be an ethylene glycol, —O—CH₂—CH₂—O—, or propylene glycol, —O—CH₂—CH₂—CH₂—O—, capped with a hydrogen at the chain termination point. Other examples of poly(alkylene glycol) include, but are not limited to, PEG, PEG derivatives such as methoxypoly(ethylene glycol) (mPEG), poly(ethylene oxide), PPG, poly(tetramethylene glycol), poly(ethylene oxide-co-propylene oxide), or copolymers and combinations thereof.

“Thiol” refers to the group —SH.

“Thioxo” or “thioketo” refers to the atom (═S).

“Alkylthio” or “thioalkoxy” refers to the group —S-alkyl, wherein alkyl is as defined herein. In certain embodiments, sulfur may be oxidized to —S(O)—. The sulfoxide may exist as one or more stereoisomers.

“Thioaryloxy” refers to the group aryl-S— wherein the aryl group is as defined herein including optionally substituted aryl groups also defined herein.

“Thioheteroaryloxy” refers to the group heteroaryl-S— wherein the heteroaryl group is as defined herein including optionally substituted aryl groups as also defined herein.

“Thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —S(O)R⁷⁰, —SO₂R⁷⁰, —SO₂O⁻M⁺, —SO₂OR⁷⁰, —OSO₂R⁷⁰, —OSO₂O−M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heterocycloalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R^{80′}s, taken together with the nitrogen atom to which they are bonded, form a 3-, 4-, 5-, 6-, or 7-membered heterocycloalkyl which may optionally include from 1 to 4 of the same or different additional heteroatoms selected from the group consisting of O, N and S, of which N may have —H, C₁-C₄ alkyl, —C(O)C₁₋₄alkyl, —CO₂C₁₋₄alkyl, or —SO₂C₁₋₄alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the embodiments and the other a typical counter ion such as chloride, or two ionized compounds disclosed herein can serve as counter ions for such divalent alkali earth ions, or a doubly ionized compound of the embodiments can serve as the counter ion for such divalent alkali earth ions). As specific examples, —NR⁸⁰R⁸⁰ is meant to include —NH₂, —NH-alkyl, N-pyrrolidinyl, N-piperazinyl, 4-N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —S(O)R⁷⁰, —SO₂R⁷⁰, —SO₃−M⁺, —SO₃R⁷⁰, —OSO₂R⁷⁰, —OSO₃−M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂−M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂−M⁺, —OCO₂R⁷⁰, —OC(S)OR⁷⁰, —NR⁷⁰C(O)R⁷⁰, —NR⁷⁰C(S)R⁷⁰, —NR⁷⁰CO₂−M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the substituent groups disclosed with respect to the individual terms herein, substituent groups for hydrogens on nitrogen atoms in “substituted” heterocycloalkyl and cycloalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)R⁷⁰, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. PET and SPECT studies may be performed as described, for example, by Brooks, D. J., “Positron Emission Tomography and Single-Photon Emission Computed Tomography in Central Nervous System Drug Development,” NeuroRx 2005, 2(2), 226-236, and references cited therein. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

### DETAILED DESCRIPTION

The present disclosure relates to aryl- and heteroaryl-substituted benzene compounds, pharmaceutical compositions containing them, and methods of using them, including methods for modulating, activating, increasing or stimulating autophagy by preventing, reversing, slowing or inhibiting the PI3K-AKT-MTOR pathway, and methods of treating diseases that are associated with regulating autophagy.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, 4^{th} edition, New York: Oxford University Press, 2002, pp. 360-361, 1084-1085; Smith and March, March's Advanced Organic Chemistry: Reactions, Mechanisms, and Structure, 5^{th} edition, Wiley-Interscience, 2001.

The nomenclature used herein to name the subject compounds is illustrated in the Examples herein. This nomenclature has generally been derived using the commercially-available AutoNom software (MDL, San Leandro, Calif.).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present invention and are disclosed herein just as if each and every such sub-combination of chemical groups was individually and explicitly disclosed herein.

### Representative Embodiments

### Formula (I)

In some embodiments of Formula (I), R¹, R², R³, and R⁴ are each independently hydrogen, hydroxy, halogen, C₁₋₄ alkyl, substituted C₁₋₄ alkyl, C₁₋₄ alkoxy, or substituted C₁₋₄ alkoxy. In certain instances, for each of R¹, R², R³, and R⁴, substituted C₁₋₄ alkyl and substituted C₁₋₄ alkoxy groups are substituted with one or more substituents selected from the group consisting of hydroxyl, halogen, —NR^{f}R^{g}, cyano, nitro, C₁₋₄ alkoxy, and C₁₋₄ haloalkoxy, wherein R^{f} and R^{g} are each independently H, C₁₋₄alkyl, —COC₁₋₄alkyl, —CO₂C₁₋₄alkyl, or —SO₂C₁₋₄alkyl.

In certain instances, R¹ is hydrogen. In certain instances, R¹ is hydroxyl. In certain instances, R¹ is halogen. In certain instances, R¹ is chloro.

In certain instances, R² is hydrogen. In certain instances, R² is hydroxyl. In certain instances, R² is halogen. In certain instances, R² is C₁₋₄ alkyl or substituted C₁₋₄ alkyl. In certain instances, R² is C₁₋₄ alkoxy or substituted C₁₋₄ alkoxy. In certain instances, R² is hydrogen or halogen, or is C₁₋₄alkyl or C₁₋₄alkoxy, each unsubstituted or substituted with at least one halogen substituent. In certain instances, R² is —CF₃, chloro, or hydrogen. In certain instances, R² is —OCF₃.

In certain instances, R³ is hydrogen. In certain instances, R³ is hydroxyl. In certain instances, R³ is halogen. In certain instances, R³ is C₁₋₄ alkyl or substituted C₁₋₄ alkyl. In certain instances, R³ is C₁₋₄ alkoxy or substituted C₁₋₄ alkoxy. In certain instances, R³ is C₁₋₄alkyl or C₁₋₄alkoxy, each unsubstituted or substituted with at least one substituent selected from the group consisting of hydroxyl, halogen, amino, cyano, and nitro. In certain instances, R³ is hydrogen or halogen, or is C₁₋₄alkyl or C₁₋₄alkoxy, each unsubstituted or substituted with at least one halogen substituent. In certain instances, R³ is chloro, —CF₃, —OCF₃, or fluoro.

In certain instances, R⁴ is hydrogen. In certain instances, R⁴ is hydroxyl. In certain instances, R⁴ is halogen.

In certain instances, R¹ and R⁴ are hydrogen. In certain instances, R² and R³ are each independently selected from hydrogen, fluoro, chloro, bromo, iodo, trifluoromethyl, trifluoromethoxy, methoxy, and hydroxyl. In certain instances, R² and R³ are each independently selected from hydrogen, fluoro, chloro, trifluoromethyl, and trifluoromethoxy.

In some embodiments of Formula (I), X is absent. In certain instances, X is C₁₋₆ alkylene, wherein one carbon unit of said alkylene is optionally replaced with —O—, —S—, —NR^{a}—, —SO—. —SO₂—, or —CO—; wherein Ra is hydrogen or C₁₋₄ alkyl. In certain instances, X is C₁₋₆ alkylene. In certain instances, X is C₁₋₄ alkylene, wherein one carbon unit of said alkylene is optionally replaced as described above. In certain instances, X is C₁₋₂ alkylene, wherein one carbon unit of said alkylene is optionally replaced as described above. In certain instances, X is C₁₋₄ alkylene. In certain instances, X is —O—. In certain instances, X is —NR^{a}—, wherein R^{a} is hydrogen or C₁₋₄ alkyl. In certain instances, X is —SO₂—. In certain instances, X is —CO—. In certain instances, X is absent or selected from C₁₋₃ alkylene, —O—, —NR^{a}—, —SO₂—, and —CO—. In certain instances, X is absent or is —SO₂—, —O—, —NH—, —CH₂—, or —CO—. In certain instances, X is —SO₂—, —O—, —NH—, —CH₂—, or —CO—.

In Formula (I), G⁴, G⁵, G⁶, and G⁷ are each independently CR¹⁰ or N. In certain instances, G⁴, G⁵, G⁶, and G⁷ are each CH. In certain instances, G⁴, G⁵, G⁶, and G⁷ are each N. In certain instances, G⁴ is N; and G⁵, G⁶, and G⁷ are each CH. In certain instances, G⁴ and G⁵ are each N; and G⁶ and G⁷ are each CH. In certain instances, G⁴ and G⁶ are each N; and G⁵ and G⁷ are each CH. In certain instances, G⁴, G⁵, G⁶ are each N; and G⁷ is CH. In certain instances, any of G⁴, G⁵, G⁶, and G⁷ are CR¹⁰, wherein each R¹⁰ is independently hydrogen, hydroxy, halogen, C₁₋₄ alkyl, C₁₋₄haloalkyl, C₁₋₄ alkoxy, or C₁₋₄haloalkoxy.

In some embodiments of Formula (I), Y is absent. In certain instances, Y is C₁₋₆ alkylene, wherein one carbon unit of said alkylene is optionally replaced with —O—, —S—, —NH—, —SO—, —SO₂—, —CO—, —CO₂—, —CONH—, —NHCO—, or —NHSO₂—, —SO₂NH—. In certain instances, Y is C₁₋₃ alkylene, wherein one carbon unit of said alkylene is optionally replaced with —O—, —S—, —NH—, —SO—, —SO₂—, —CO—, —CO₂—, —CONH—, —NHCO—, or —NHSO₂—, —SO₂NH—. In certain instances, Y is C₁₋₃ alkylene. In certain instances, Y is —O—. In certain instances, Y is —NH—. In certain instances, Y is —SO₂. In certain instances, Y is —CO—. In certain instances, Y is —CO₂—. In certain instances, Y is —CONH—. In certain instances, Y is —NHCO—. In certain instances, Y is —NHSO₂—. In certain instances, Y is —SO₂NH—. In certain instances, Y is selected from —O—, —S—, —NH—, —SO—, —SO₂—, —CO—, —CO₂—, —CONH—, —NHCO—, —NHSO₂—, and —SO₂NH—. In certain instances, Y is selected from —O—, —NH—, —SO₂—, and —CO—.

In some embodiments of Formula (I), Ring A is a 5-membered heteroaryl ring containing one, two, three, or four heteroatoms. In certain instances, Ring A contains one heteroatom. In certain instances, Ring A contains two heteroatoms. In certain instances, Ring A contains three heteroatoms. In certain instances, Ring A contains four heteroatoms. In certain instances, Ring A contains at least one heteroatom selected from nitrogen, sulfur, and oxygen. In certain instances, Ring A contains carbon, nitrogen, and sulfur ring members. In certain instances, Ring A contains carbon and nitrogen ring members. In certain instances, Ring A contains carbon, nitrogen, and oxygen ring members.

In certain instances, Ring A is furanyl, thiophenyl, pyrrolyl, oxazolyl, thiazolyl, imidazolyl, thiadiazolyl, oxadiazolyl, triazolyl, or tetrazolyl, each optionally substituted with —(R⁵)ₙ as described for Ring A. In certain instances, Ring A is thiadiazolyl, thiazolyl, triazolyl, oxadiazoyl, tetrazolyl, imidazolyl, or pyrrolyl, each optionally substituted with —(R⁵)ₙ as described for Ring A. In certain instances, Ring A is selected from the following:

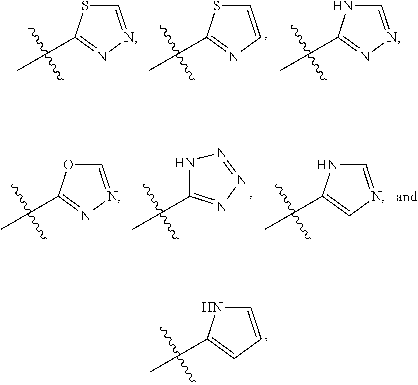

each optionally substituted with —(R⁵)ₙ as described for Ring A. In certain instances, Ring A is selected from the following:

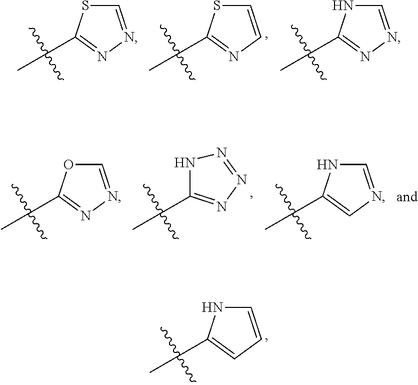

where n is zero. In certain instances, Ring A is

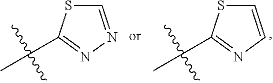 [SMILES: *c1nncs1 ; "or" ; *c1nccs1 ; ","]

each unsubstituted or substituted with —(R⁵)ₙ as described for Ring A. In certain instances, Ring A is

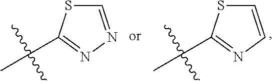

where n is zero.

In certain instances, R⁵ is C₁₋₆ alkyl or substituted C₁₋₆ alkyl. In certain instances, R⁵ is C₁₋₆ alkoxy or substituted C₁₋₆ alkoxy. In certain instances, R⁵ is C₃₋₈ cycloalkyl or substituted C₃₋₈ cycloalkyl. In certain instances, R⁵ is C₃₋₈ cycloalkoxy or substituted C₃₋₈ cycloalkoxy. In certain instances, R⁵ is hydroxyl. In certain instances, R⁵ is halogen. In certain instances, R⁵ is bromo. In certain instances, R⁵ is —NR^{m}Rⁿ, where R^{m} and Rⁿ are each independently H or C₁₋₄alkyl. In certain instances, R⁵ is cyano.

In certain instances, where R⁵ is an alkyl, alkoxy, cycloalkyl, or cycloalkoxy that is substituted, R⁵ is substituted with at least one substituent selected from the group consisting of hydroxyl, halogen, —NR^{b}R^{c}, optionally substituted heterocycloalkyl, poly(alkylene glycol), C₁₋₄ alkoxy, C₁₋₄ haloalkoxy, —CO₂H, —CO₂C₁₋₄alkyl, or —OC(O)C₁₋₄alkyl, wherein R^{b} and R^{c} are each independently hydrogen, optionally substituted —C₁₋₄alkyl, —COC₁₋₄alkyl, —SO₂C₁₋₄alkyl, or —CO₂C₁₋₄alkyl; or R^{b} and R^{c} taken together with the nitrogen to which they are attached form a monocyclic heterocycloalkyl, wherein the monocyclic heterocycloalkyl is unsubstituted or substituted with C₁₋₄alkyl, —OH, amino, C₁₋₄alkylamino, —SO₂C₁₋₄alkyl, —COC₁₋₄alkyl, or —CO₂C₁₋₄alkyl.

In certain instances, R⁵ is C₁₋₄alkyl substituted with poly(alkylene glycol). In certain instances, the poly(alkylene glycol) comprises one to 10 alkylene glycol subunits. In certain instances, the poly(alkylene glycol) comprises one to 5 alkylene glycol subunits. In certain instances, the poly(alkylene glycol) comprises one or 2 alkylene glycol subunits. In certain instances, R⁵ is substituted with —O(CH₂)₂₋₃—(CH₂)₂₋₃—OH or —O(CH₂)₂₋₃—(CH₂)₂₋₃—OC₁₋₄alkyl.

In certain instances, R⁵ is C₁₋₄alkyl substituted with hydroxyl, —NR^{b}R^{c}, an optionally substituted heterocycloalkyl, or poly(alkylene glycol). In certain instances, R^{b} and R^{c} are independently hydrogen or a C₁₋₄alkyl unsubstituted or substituted with hydroxyl, halogen, C₁₋₄alkoxy, —COC₁₋₄alkyl, —CO₂C₁₋₄alkyl, or —SO₂C₁₋₄alkyl. In certain instances, R^{b} and R^{c} taken together with the nitrogen to which they are attached form an aziridine, pyrrolidine, piperidine, piperazine, morpholine, thiomorpholine, or azepine ring, each ring optionally substituted with C₁₋₄alkyl, amino, hydroxyl, —SO₂C₁₋₄alkyl, —COC₁₋₄alkyl, or —CO₂C₁₋₄alkyl. In certain instances, R⁵ is C₁₋₄alkyl substituted with a monocyclic carbon-linked heterocycloalkyl ring, unsubstituted or substituted with C₁₋₄alkyl, —COC₁₋₄alkyl, —CO₂C₁₋₄alkyl, or —SO₂C₁₋₄alkyl. In certain instances, R⁵ is (dimethylamino)methyl, aziridin-1-ylmethyl, piperidin-1-ylmethyl, 4-methylpiperazin-1-ylmethyl, morpholinomethyl, 1-hydroxyethyl, aminomethyl, acetamidomethyl, methanesulfonamidomethyl, or (4-(methylsulfonyl)piperazin-1-yl)methyl.

In some embodiments of Formula (I), n is zero. In certain instances, n is zero or one. In certain instances, n is one. In certain instances, n is 2. In certain instances, n is 3.

### Formula (II)

The present disclosure provides compounds of Formula (II), shown herein,

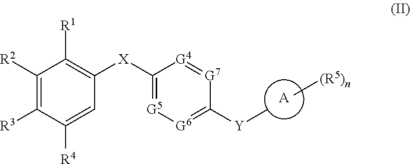

wherein
R¹, R², R³, and R⁴ are each independently hydrogen, hydroxy, halogen, C₁₋₄ alkyl, or C₁₋₄ alkoxy, wherein each alkyl or alkoxy is unsubstituted or substituted with one or more substituents independently selected from hydroxy, halogen, amino, cyano, and nitro;
X is absent, or is C₁₋₆ alkylene, wherein one carbon unit of said alkylene is optionally replaced with —O—, —S—, —SO—, —NR^{a}—, —SO₂—, or —CO—;
   wherein R^{a} is hydrogen or C₁₋₄ alkyl;
G⁴, G⁵, G⁶, and G⁷ are each independently CR¹⁰ or N;
   wherein each R¹⁰ is independently hydrogen, hydroxy, halogen, C₁₋₄ alkyl, C₁₋₄ haloalkyl, C₁₋₄ alkoxy, or C₁₋₄ haloalkoxy;
Y is absent, or is C₁₋₆ alkylene, wherein one carbon unit of said alkylene is optionally replaced with —O—, —S—, —NH—, —SO—, —SO₂—, —CO—, —CO₂—, —CONH—, —NHCO—, —NHSO₂—, or —SO₂NH—;
Ring A is a 5-membered heteroaryl ring;
each R⁵ is independently C₁₋₆ alkyl, C₁₋₆ alkoxy, C₃₋₈ cycloalkyl, C₃₋₈ cycloalkoxy, hydroxyl, halogen, —NR^{m}Rⁿ, or cyano;
   wherein R^{m} and Rⁿ are each independently H or C₁₋₄alkyl; and
   each alkyl, alkoxy, cycloalkyl, or cycloalkoxy is unsubstituted or substituted with hydroxyl, halogen, —NR^{b}R^{c}, monocyclic heterocycloalkyl, or poly(alkylene glycol);
      wherein said monocyclic heterocycloalkyl is unsubstituted or substituted with C₁₋₄alkyl, —SO₂C₁₋₄alkyl, —COC₁₋₄alkyl, or —CO₂C₁₋₄alkyl;
      wherein R^{b} and R^{c} are each independently hydrogen, —C₁₋₄alkyl, —COC₁₋₄alkyl, —SO₂C₁₋₄alkyl, or —CO₂C₁₋₄alkyl;
         wherein each alkyl is unsubstituted or substituted with hydroxyl, C₁₋₄alkoxy, halogen, or —SO₂C₁₋₄alkyl;
      or R^{b} and R^{c} taken together with the nitrogen to which they are attached form a monocyclic heterocycloalkyl,
         wherein the monocyclic heterocycloalkyl is unsubstituted or substituted with C₁₋₄alkyl, —SO₂C₁₋₄alkyl, —COC₁₋₄alkyl, or —CO₂C₁₋₄alkyl; and
n is a number from zero to three;
   or a pharmaceutically acceptable salt thereof.

In some embodiments of Formula (II), R¹ is hydrogen. In certain instances, R¹ is hydroxyl. In certain instances, R¹ is halogen. In certain instances, R¹ is chloro.

In certain instances, R² is hydrogen. In certain instances, R² is hydroxyl. In certain instances, R² is halogen. In certain instances, R² is C₁₋₄ alkyl or substituted C₁₋₄ alkyl. In certain instances, R² is C₁₋₄ alkoxy or substituted C₁₋₄ alkoxy. In certain instances, R² is hydrogen or halogen, or is C₁₋₄alkyl or C₁₋₄alkoxy, each unsubstituted or substituted with at least one halogen substituent. In certain instances, R₂ is —CF₃, chloro, or hydrogen. In certain instances, R₂ is —OCF₃.

In certain instances, R³ is hydrogen. In certain instances, R³ is hydroxyl. In certain instances, R³ is halogen. In certain instances, R³ is C₁₋₄ alkyl or substituted C₁₋₄ alkyl. In certain instances, R³ is C₁₋₄ alkoxy or substituted C₁₋₄ alkoxy. In certain instances, R³ is C₁₋₄alkyl or C₁₋₄alkoxy, each unsubstituted or substituted with at least one substituent selected from the group consisting of hydroxyl, halogen, amino, cyano, and nitro. In certain instances, R³ is hydrogen or halogen, or is C₁₋₄alkyl or C₁₋₄alkoxy, each unsubstituted or substituted with at least one halogen substituent. In certain instances, R³ is chloro, —CF₃, —OCF₃, or fluoro.

In certain instances, R⁴ is hydrogen. In certain instances, R⁴ is hydroxyl. In certain instances, R⁴ is halogen.

In certain instances, R¹ and R⁴ are hydrogen. In certain instances, R² and R³ are independently selected from hydrogen, fluoro, chloro, bromo, iodo, trifluoromethyl, trifluoromethoxy, methoxy, and hydroxyl. In certain instances, R² and R³ are independently selected from hydrogen, fluoro, chloro, trifluoromethyl, and trifluoromethoxy.

In some embodiments of Formula (II), X is absent. In certain instances, X is C₁₋₆ alkylene, wherein one carbon unit of said alkylene is optionally replaced with —O—, —S—, —NR^{a}—, —SO—, —SO₂—, or —CO—; wherein R^{a} is hydrogen or C₁₋₄ alkyl. In certain instances, X is C₁₋₆ alkylene. In certain instances, X is C₁₋₄ alkylene, wherein one carbon unit of said alkylene is optionally replaced as described above. In certain instances, X is C₁₋₃ alkylene, wherein one carbon unit of said alkylene is optionally replaced as described above. In certain instances, X is C₃₋₆ alkylene. In certain instances, X is —O—. In certain instances, X is —NR^{a}—, wherein R^{a} is hydrogen or C₁₋₄ alkyl. In certain instances, X is —SO₂—. In certain instances, X is —CO—. In certain instances, X is absent or selected from C₁₋₃ alkylene, —O—, —NR^{a}—, —SO₂—, and —CO—. In certain instances, X is absent or is —SO₂—, —O—, —NH—, —CH₂—, or —CO—. In certain instances, X is —SO₂—, —O—, —NH—, —CH₂—, or —CO—.

In some embodiments of Formula (II), G⁴, G⁵, G⁶, and G⁷ are each CH. In certain instances, G⁴, G⁵, G⁶, and G⁷ are each N. In certain instances, G⁴ is N; and G⁵, G⁶, and G⁷ are each CH. In certain instances, G⁴ and G⁵ are each N; and G⁶ and G⁷ are each CH. In certain instances, G⁴ and G⁶ are each N; and G⁵ and G⁷ are each CH. In certain instances, G⁴, G⁵, G⁶ are each N; and G⁷ is CH. In certain instances, any of G⁴, G⁵, G⁶, and G⁷ are CR¹⁰, wherein R¹⁰ is independently hydrogen, hydroxy, halogen, C₁₋₄ alkyl, C₁₋₄ haloalkyl, C₁₋₄ alkoxy, or C₁₋₄ haloalkoxy.

In some embodiments of Formula (II), Y is absent. In certain instances, Y is C₁₋₆ alkylene, wherein one carbon unit of said alkylene is optionally replaced with —O—, —S—, —NH—, —SO—, —SO₂—, —CO—, —CO₂—, —CONH—, —NHCO—, or —NHSO₂—, —SO₂NH—. In certain instances, Y is C₁₋₃ alkylene, wherein one carbon unit of said alkylene is optionally replaced as described above. In certain instances, Y is C₁₋₃ alkylene. In certain instances, Y is —O—. In certain instances, Y is —NH—. In certain instances, Y is —SO₂. In certain instances, Y is —CO—. In certain instances, Y is —CO₂—. In certain instances, Y is —CONH—. In certain instances, Y is —NHCO—. In certain instances, Y is —NHSO₂—. In certain instances, Y is —SO₂NH—. In certain instances, Y is selected from —O—, —NH—, —SO₂—, —CO—, —CO₂—, —CONH—, —NHCO—, —NHSO₂—, and —SO₂NH—. In certain instances, Y is selected from —O—, —NH—, —SO₂—, and —CO—.

In some embodiments of Formula (II), Ring A is a 5-membered heteroaryl ring containing one, two, three, or four heteroatoms. In certain instances, Ring A contains one heteroatom. In certain instances, Ring A contains two heteroatoms. In certain instances, Ring A contains three heteroatoms. In certain instances, Ring A contains four heteroatoms. In certain instances, Ring A contains at least one heteroatom selected from nitrogen, sulfur, and oxygen. In certain instances, Ring A contains carbon, nitrogen, and sulfur ring members. In certain instances, Ring A contains carbon and nitrogen ring members. In certain instances, Ring A contains carbon, nitrogen, and oxygen ring members.

In certain instances, Ring A is furanyl, thiophenyl, pyrrolyl, oxazolyl, thiazolyl, imidazolyl, thiadiazolyl, oxadiazolyl, triazolyl, or tetrazolyl, each optionally substituted with —(R⁵)ₙ as described for Ring A. In certain instances, Ring A is thiadiazolyl, thiazolyl, triazolyl, oxadiazoyl, tetrazolyl, imidazolyl, or pyrrolyl, each optionally substituted with —(R⁵)ₙ as described for Ring A. In certain instances, Ring A is selected from the following:

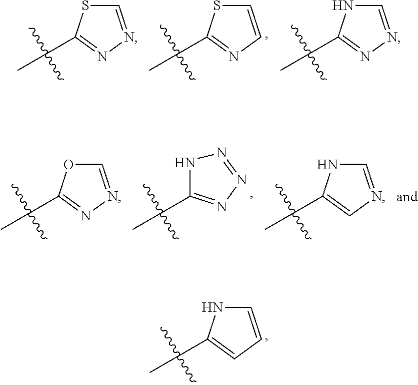

each optionally substituted with —(R⁵)ₙ as described for Ring A. In certain instances, Ring A is selected from the following:

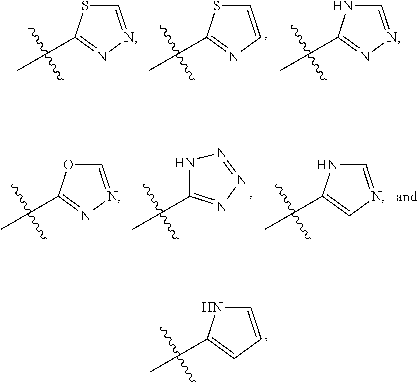

where n is zero. In certain instances, Ring A is

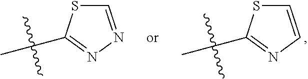 [SMILES: *c1nncs1 ; *c1nccs1]

each unsubstituted or substituted with —(R⁵)ₙ as described for Ring A. In certain instances, Ring A is

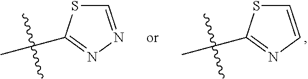

where n is zero.

In some embodiments of Formula (II), R⁵ is C₁₋₆ alkyl or substituted C₁₋₆ alkyl. In certain instances, R⁵ is C₁₋₆alkoxy or substituted C₁₋₆alkoxy. In certain instances, R⁵ is C₃₋₈ cycloalkyl or substituted C₃₋₈ cycloalkyl. In certain instances, R⁵ is C₃₋₈ cycloalkoxy or substituted C₃₋₈ cycloalkoxy. In certain instances, R⁵ is hydroxyl. In certain instances, R⁵ is halogen. In certain instances, R⁵ is bromo. In certain instances, R⁵ is —NR^{m}Rⁿ, where R^{m} and IV are each independently H or C₁₋₄alkyl. In certain instances, R⁵ is cyano.

In certain instances, the substituent on R⁵ is hydroxyl. In certain instances, the substituent on R⁵ is —NR^{b}R^{c}. In certain instances, the substituent on R⁵ is an unsubstituted or substituted monocyclic heterocycloalkyl.

In certain instances, R⁵ is C₁₋₄alkyl substituted with poly(alkylene glycol). In certain instances, the poly(alkylene glycol) comprises one to 10 alkylene glycol subunits. In certain instances, the poly(alkylene glycol) comprises one to 5 alkylene glycol subunits. In certain instances, the poly(alkylene glycol) comprises one or 2 alkylene glycol subunits. In certain instances, R⁵ is substituted with —O(CH₂)₂₋₃—(CH₂)₂₋₃—OH or —O(CH₂)₂₋₃—(CH₂)₂₋₃—OC₁₋₄alkyl.

In certain instances, R⁵ is C₁₋₄alkyl substituted with hydroxyl, —NR^{b}R^{c}, an optionally substituted monocyclic heterocycloalkyl, or a poly(alkylene glycol). In certain instances, R⁵ is substituted C₁₋₄ alkyl and the substituent on R⁵ is hydroxyl. In certain instances, R⁵ is substituted C₁₋₄ alkyl and the substituent on R⁵ is —NR^{b}R^{c}. In certain instances, R⁵ is substituted C₁₋₄ alkyl and the substituent on R⁵ is an unsubstituted or substituted monocyclic heterocycloalkyl. In certain instances, R⁵ is substituted C₁₋₄ alkyl and the substituent on R⁵ is a poly(alkylene glycol).

In some embodiments of Formula (II), R⁵ is C₁₋₄alkyl substituted with hydroxyl or —NR^{b}R^{c}. In certain instances, R^{b} and R^{c} are independently hydrogen or a C₁₋₄alkyl unsubstituted or substituted with hydroxyl or —SO₂C₁₋₄alkyl. In certain instances, R^{b} and R^{c} taken together with the nitrogen to which they are attached form an aziridine, pyrrolidine, piperidine, piperazine, morpholine, or azepine ring, each optionally substituted with C₁₋₄alkyl, —SO₂C₁₋₄alkyl, —COC₁₋₄alkyl, or —CO₂C₁₋₄alkyl. In certain instances, R⁵ is C₁₋₄alkyl substituted with a monocyclic carbon-linked heterocycloalkyl ring, unsubstituted or substituted with C₁₋₄alkyl, —COC₁₋₄alkyl, —CO₂C₁₋₄alkyl, or —SO₂C₁₋₄alkyl. In certain instances, R⁵ is (dimethylamino)methyl, aziridin-1-ylmethyl, piperidin-1-ylmethyl, 4-methylpiperazin-1-ylmethyl, morpholinomethyl, 1-hydroxyethyl, aminomethyl, acetamidomethyl, methanesulfonamidomethyl, or (4-(methylsulfonyl)piperazin-1-yl)methyl.

In certain instances, n is one and R⁵ is substituted C₁₋₄ alkyl and the substituent on R⁵ is hydroxyl or —NR^{b}R^{c}. In certain instances, n is one and R⁵ is substituted C₁₋₆ alkyl and the substituent on R⁵ is —NR^{b}R^{c} and R^{b} and R^{c} are each independently hydrogen or —C₁₋₄alkyl. In certain instances, R^{b} and R^{c} are each hydrogen. In certain instances, R^{b} and R^{c} are each —C₁₋₄alkyl. In certain instances, n is one and R⁵ is substituted C₁₋₄ alkyl and the substituent on R⁵ is —NR^{b}R^{c} and R^{b} and R^{c} are taken together with the nitrogen to which they are attached to form a monocyclic heterocycloalkyl, wherein the monocyclic heterocycloalkyl is unsubstituted or substituted with C₁₋₄alkyl, —SO₂C₁₋₄alkyl, —COC₁₋₄alkyl, or —CO₂C₁₋₄alkyl. In certain instances, the monocyclic heterocycloalkyl is substituted with C₁₋₄alkyl or —SO₂C₁₋₄alkyl. In certain instances, n is one and R⁵ is (dimethylamino)methyl, aziridin-1-ylmethyl, piperidin-1-ylmethyl, 4-methylpiperazin-1-ylmethyl, morpholinomethyl, 1-hydroxyethyl, aminomethyl, acetamidomethyl, methanesulfonamidomethyl, or (4-(methylsulfonyl)piperazin-1-yl)methyl.

In some embodiments of Formula (II), n is zero. In certain instances, n is zero or one. In certain instances, n is one. In certain instances, n is 2. In certain instances, n is 3.

### Formula (III)

The present disclosure provides compounds of Formula (III), shown herein.

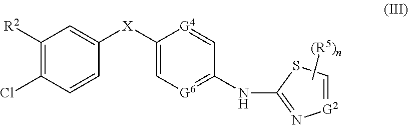

wherein

### R² is H or —CF₃;

### X is —SO₂—, —O—, —NH—, or —CO—;

G², G⁴, and G⁶ are each independently CH or N;
R⁵ is C₁₋₄alkyl optionally substituted with —NR^{b}R^{c};
wherein R^{b} and R^{c} are each independently H or C₁₋₄alkyl; or R^{b} and R^{c} taken together with the nitrogen to which they are attached form a monocyclic heterocycloalkyl ring, unsubstituted or substituted with C₁₋₄alkyl; and
   n is zero or one;
   or a pharmaceutically acceptable salt thereof.

In some embodiments of Formula (III), R² is H. In other embodiments, R² is —CF₃.

In some embodiments of Formula (III), X is —SO₂—. In other embodiments, X is —O—. In still other embodiments, X is —NH—. In still other embodiments, X is —CO—.

In some embodiments of Formula (III), G⁴ and G⁶ are both CH. In other embodiments, G⁴ and G⁶ are both N. In some embodiments of Formula (III), G² is N. In other embodiments, G² is CH.

In some embodiments of Formula (III), R⁵ is C₁₋₄alkyl optionally substituted with —NR^{b}R^{c}; wherein R^{b} and R^{c} are each independently H or C₁₋₄alkyl. In other embodiments, R^{b} and R^{c} taken together with the nitrogen to which they are attached form a monocyclic heterocycloalkyl ring, unsubstituted or substituted with C₁₋₄alkyl.

In some embodiments of Formula (III), n is zero. In other embodiments, n is one.

Particular compounds of interest are shown in the following table.

| Ex. | Structure | Name |
|---|---|---|
| 1 | 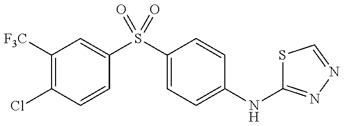 [SMILES: O=S(=O)(c1ccc(Nc2nncs2)cc1)c1ccc(Cl)c(C(F)(F)F)c1] | N-(4-((4-chloro-3- (trifluoromethyl)phenyl)sulfonyl)phe- nyl)-1,3,4-thiadiazol-2-amine; |
| 2 | 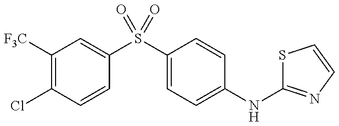 [SMILES: O=S(=O)(c1ccc(Nc2nccs2)cc1)c1ccc(Cl)c(C(F)(F)F)c1] | N-(4-((4-chloro-3- (trifluoromethyl)phenyl)sulfonyl)phe- nyl)thiazol-2-amine; |
| 3 | 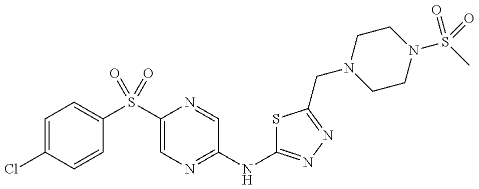 [SMILES: CS(=O)(=O)N1CCN(Cc2nnc(Nc3cnc(S(=O)(=O)c4ccc(Cl)cc4)cn3)s2)CC1] | N-(5-((4- chlorophenyl)sulfonyl)pyrazin-2-yl)- 5-((4-(methylsulfonyl)piperazin-1- yl)methyl)-1,3,4-thiadiazol-2-amine; |
| 4 | 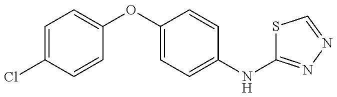 [SMILES: Clc1ccc(Oc2ccc(Nc3nncs3)cc2)cc1] | N-(4-(4-chlorophenoxy)phenyl)- 1,3,4-thiadiazol-2-amine; |
| 5 | 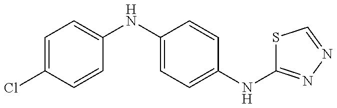 [SMILES: Clc1ccc(Nc2ccc(Nc3nncs3)cc2)cc1] | N¹-(4-chlorophenyl)-N⁴-(1,3,4- thiadiazol-2-yl)benzene-1,4-diamine; |
| 6 | 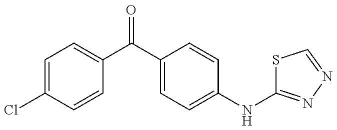 [SMILES: O=C(c1ccc(Cl)cc1)c1ccc(Nc2nncs2)cc1] | (4-((1,3,4-thiadiazol-2- yl)amino)phenyl)(4- chlorophenyl)methanone; |
| 7 | 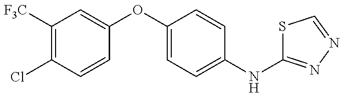 [SMILES: FC(F)(F)c1cc(Oc2ccc(Nc3nncs3)cc2)ccc1Cl] | N-(4-(4-chloro-3- (trifluoromethyl)phenoxy)phenyl)- 1,3,4-thiadiazol-2-amine; |
| 8 | 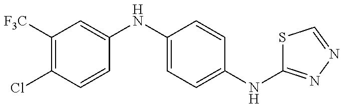 [SMILES: FC(F)(F)c1cc(Nc2ccc(Nc3nncs3)cc2)ccc1Cl] | N¹-(4-chloro-3- (trifluoromethyl)phenyl)-N⁴-(1,3,4- thiadiazol-2-yl)benzene-1,4-diamine; |
| 9 | 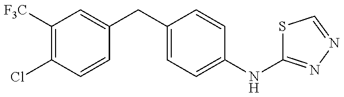 [SMILES: FC(F)(F)c1cc(Cc2ccc(Nc3nncs3)cc2)ccc1Cl] | N-(4-(4-chloro-3- (trifluoromethyl)benzyl)phenyl)- 1,3,4-thiadiazol-2-amine; |
| 10 | 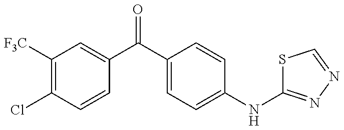 [SMILES: O=C(c1ccc(Nc2nncs2)cc1)c1ccc(Cl)c(C(F)(F)F)c1] | (4-((1,3,4-thiadiazol-2- yl)amino)phenyl)(4-chloro-3- (trifluoromethyl)phenyl)methanone; |
| 11 | 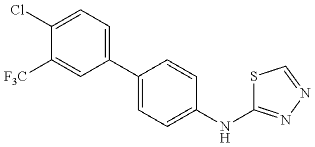 [SMILES: FC(F)(F)c1cc(-c2ccc(Nc3nncs3)cc2)ccc1Cl] | N-(4′-chloro-3′-(trifluoromethyl)- [1,1′-biphenyl]-4-yl)-1,3,4-thiadiazol- 2-amine; |
| 12 | 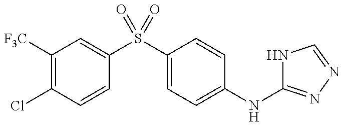 [SMILES: O=S(=O)(c1ccc(Nc2nnc[nH]2)cc1)c1ccc(Cl)c(C(F)(F)F)c1] | N-(4-((4-chloro-3- (trifluoromethyl)phenyl)sulfonyl)phe- nyl)-4H-1,2,4-triazol-3-amine; |
| 13 | 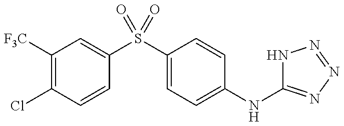 [SMILES: O=S(=O)(c1ccc(Nc2nnn[nH]2)cc1)c1ccc(Cl)c(C(F)(F)F)c1] | N-(4-((4-chloro-3- (trifluoromethyl)phenyl)sulfonyl)phe- nyl)-1H-tetrazol-5-amine; |
| 14 | 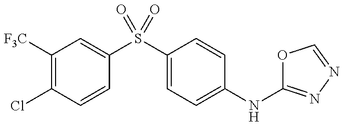 [SMILES: O=S(=O)(c1ccc(Nc2nnco2)cc1)c1ccc(Cl)c(C(F)(F)F)c1] | N-(4-((4-chloro-3- (trifluoromethyl)phenyl)sulfonyl)phe- nyl)-1,3,4-oxadiazol-2-amine; |
| 15 | 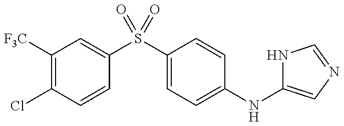 [SMILES: O=S(=O)(c1ccc(Nc2cnc[nH]2)cc1)c1ccc(Cl)c(C(F)(F)F)c1] | N-(4-((4-chloro-3- (trifluoromethyl)phenyl)sulfonyl)phe- nyl)-1H-imidazol-5-amine; |
| 16 | 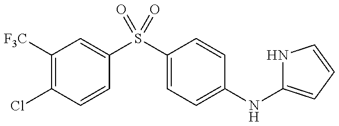 [SMILES: O=S(=O)(c1ccc(Nc2ccc[nH]2)cc1)c1ccc(Cl)c(C(F)(F)F)c1] | N-(4-((4-chloro-3- (trifluoromethyl)phenyl)sulfonyl)phe- nyl)-1H-pyrrol-2-amine; |
| 17 | 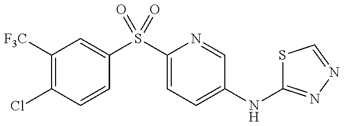 [SMILES: O=S(=O)(c1ccc(Cl)c(C(F)(F)F)c1)c1ccc(Nc2nncs2)cn1] | N-(6-((4-chloro-3- (trifluoromethyl)phenyl)sulfonyl)pyri- din-3-yl)-1,3,4-thiadiazol-2-amine; |
| 18 | 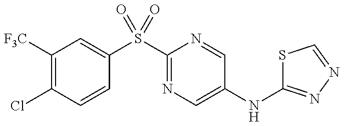 [SMILES: O=S(=O)(c1ccc(Cl)c(C(F)(F)F)c1)c1ncc(Nc2nncs2)cn1] | N-(2-((4-chloro-3- (trifluoromethyl)phenyl)sulfonyl)pyri- midin-5-yl)-1,3,4-thiadiazol-2-amine; |
| 19 | 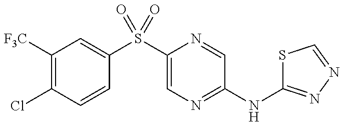 [SMILES: O=S(=O)(c1ccc(Cl)c(C(F)(F)F)c1)c1cnc(Nc2nncs2)cn1] | N-(5-((4-chloro-3- (trifluoromethyl)phenyl)sulfonyl)pyra- zin-2-yl)-1,3,4-thiadiazol-2-amine; |
| 20 | 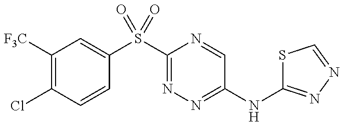 [SMILES: O=S(=O)(c1ccc(Cl)c(C(F)(F)F)c1)c1ncc(Nc2nncs2)nn1] | N-(3-((4-chloro-3- (trifluoromethyl)phenyl)sulfonyl)- 1,2,4-triazin-6-yl)-1,3,4-thiadiazol-2- amine; |
| 21 | 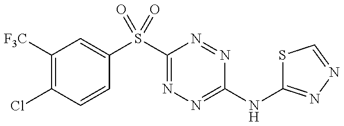 [SMILES: O=S(=O)(c1ccc(Cl)c(C(F)(F)F)c1)c1nnc(Nc2nncs2)nn1] | N-(6-((4-chloro-3- (trifluoromethyl)phenyl)sulfonyl)- 1,2,4,5-tetrazin-3-yl)-1,3,4- thiadiazol-2-amine; |
| 22 | 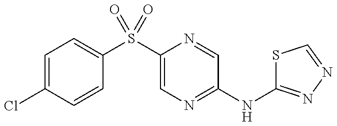 [SMILES: O=S(=O)(c1ccc(Cl)cc1)c1cnc(Nc2nncs2)cn1] | N-(5-((4- chlorophenyl)sulfonyl)pyrazin-2-yl)- 1,3,4-thiadiazol-2-amine; |
| 23 | 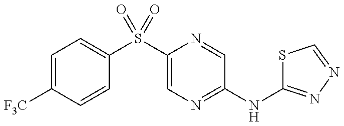 [SMILES: O=S(=O)(c1ccc(C(F)(F)F)cc1)c1cnc(Nc2nncs2)cn1] | N-(5-((4- (trifluoromethyl)phenyl)sulfonyl)pyra- zin-2-yl)-1,3,4-thiadiazol-2-amine; |
| 24 | 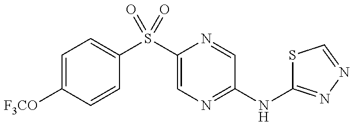 [SMILES: O=S(=O)(c1ccc(OC(F)(F)F)cc1)c1cnc(Nc2nncs2)cn1] | N-(5-((4- (trifluoromethoxy)phenyl)sulfonyl)py- razin-2-yl)-1,3,4-thiadiazol-2-amine; |
| 25 | 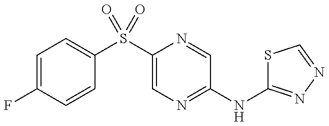 [SMILES: O=S(=O)(c1ccc(F)cc1)c1cnc(Nc2nncs2)cn1] | N-(5-((4- fluorophenyl)sulfonyl)pyrazin-2-yl)- 1,3,4-thiadiazol-2-amine; |
| 26 | 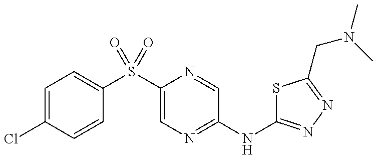 [SMILES: CN(C)Cc1nnc(Nc2cnc(S(=O)(=O)c3ccc(Cl)cc3)cn2)s1] | N-(5-((4- chlorophenyl)sulfonyl)pyrazin-2-yl)- 5-((dimethylamino)methyl)-1,3,4- thiadiazol-2-amine; |
| 27 | 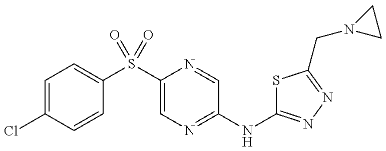 [SMILES: O=S(=O)(c1ccc(Cl)cc1)c1cnc(Nc2nnc(CN3CC3)s2)cn1] | 5-(aziridin-1-ylmethyl)-N-(5-((4- chlorophenyl)sulfonyl)pyrazin-2-yl)- 1,3,4-thiadiazol-2-amine; |
| 28 | 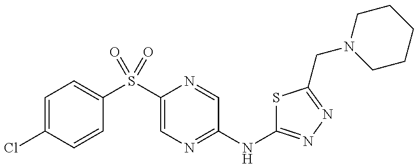 [SMILES: O=S(=O)(c1ccc(Cl)cc1)c1cnc(Nc2nnc(CN3CCCCC3)s2)cn1] | N-(5-((4- chlorophenyl)sulfonyl)pyrazin-2-yl)- 5-(piperidin-1-ylmethyl)-1,3,4- thiadiazol-2-amine; |
| 29 | 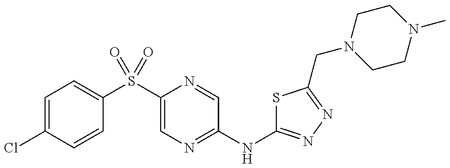 [SMILES: CN1CCN(Cc2nnc(Nc3cnc(S(=O)(=O)c4ccc(Cl)cc4)cn3)s2)CC1] | N-(5-((4- chlorophenyl)sulfonyl)pyrazin-2-yl)- 5-((4-methylpiperazin-1-yl)methyl)- 1,3,4-thiadiazol-2-amine; |
| 30 | 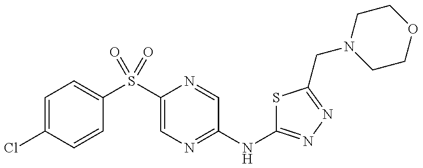 [SMILES: O=S(=O)(c1ccc(Cl)cc1)c1cnc(Nc2nnc(CN3CCOCC3)s2)cn1] | N-(5-((4- chlorophenyl)sulfonyl)pyrazin-2-yl)- 5-(morpholinomethyl)-1,3,4- thiadiazol-2-amine; |
| 31 | 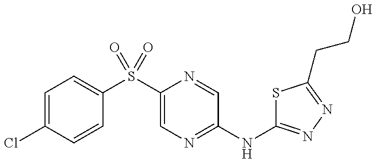 [SMILES: O=S(=O)(c1ccc(Cl)cc1)c1cnc(Nc2nnc(CCO)s2)cn1] | 2-(5-((5-((4- chlorophenyl)sulfonyl)pyrazin-2- yl)amino)-1,3,4-thiadiazol-2- yl)ethanol; |
| 32 | 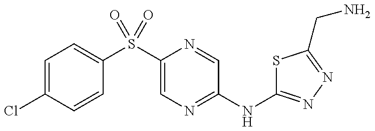 [SMILES: NCc1nnc(Nc2cnc(S(=O)(=O)c3ccc(Cl)cc3)cn2)s1] | 5-(aminomethyl)-N-(5-((4- chlorophenyl)sulfonyl)pyrazin-2-yl)- 1,3,4-thiadiazol-2-amine; |
| 33 | 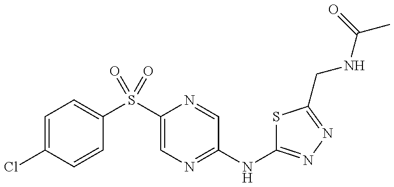 [SMILES: CC(=O)NCc1nnc(Nc2cnc(S(=O)(=O)c3ccc(Cl)cc3)cn2)s1] | N-((5-((5-((4- chlorophenyl)sulfonyl)pyrazin-2- yl)amino)-1,3,4-thiadiazol-2- yl)methyl)acetamide; and |
| 34 | 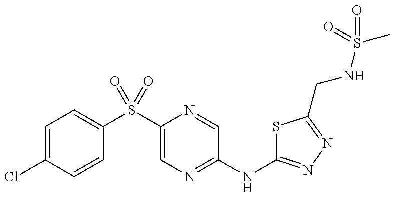 [SMILES: CS(=O)(=O)NCc1nnc(Nc2cnc(S(=O)(=O)c3ccc(Cl)cc3)cn2)s1] | N-((5-((5-((4- chlorophenyl)sulfonyl)pyrazin-2- yl)amino)-1,3,4-thiadiazol-2- yl)methyl)methanesulfonamide; |

and pharmaceutically acceptable salts thereof.

Additional compounds are shown in the following table.

| Ex. | Structure | Name |
|---|---|---|
| 35 | 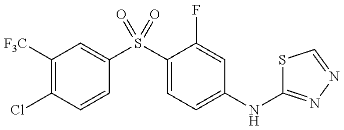 [SMILES: O=S(=O)(c1ccc(Cl)c(C(F)(F)F)c1)c1ccc(Nc2nncs2)cc1F] | N-(4-((4-chloro-3- (trifluoromethyl)phenyl)sulfonyl)-3- fluorophenyl)-1,3,4-thiadiazol-2-amine; |
| 36 | 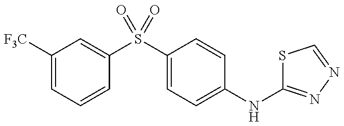 [SMILES: O=S(=O)(c1ccc(Nc2nncs2)cc1)c1cccc(C(F)(F)F)c1] | N-(4-((3- (trifluoromethyl)phenyl)sulfonyl)phenyl)- 1,3,4-thiadiazol-2-amine; |
| 37 | 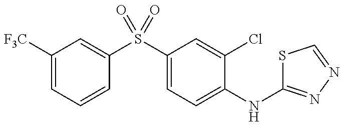 [SMILES: O=S(=O)(c1cccc(C(F)(F)F)c1)c1ccc(Nc2nncs2)c(Cl)c1] | N-(2-chloro-4-((3- (trifluoromethyl)phenyl)sulfonyl)phenyl)- 1,3,4-thiadiazol-2-amine; |
| 38 | 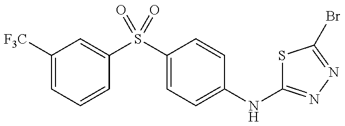 [SMILES: O=S(=O)(c1ccc(Nc2nnc(Br)s2)cc1)c1cccc(C(F)(F)F)c1] | 5-bromo-N-(4-((3- (trifluoromethyl)phenyl)sulfonyl)phenyl)- 1,3,4-thiadiazol-2-amine; |
| 39 | 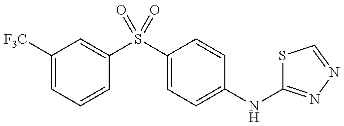 [SMILES: O=S(=O)(c1ccc(Nc2nncs2)cc1)c1cccc(C(F)(F)F)c1] | N-(4-((3- (trifluoromethoxy)phenyl)sulfonyl)phenyl)- 1,3,4-thiadiazol-2-amine; |
| 40 | 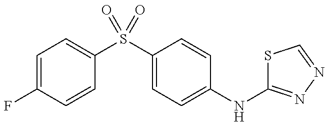 [SMILES: O=S(=O)(c1ccc(F)cc1)c1ccc(Nc2nncs2)cc1] | N-(4-((4-fluorophenyl)sulfonyl)phenyl)-1,3,4- thiadiazol-2-amine; and |
| 41 | 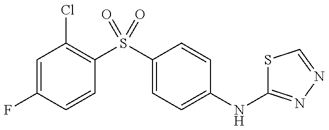 [SMILES: O=S(=O)(c1ccc(Nc2nncs2)cc1)c1ccc(F)cc1Cl] | N-(4-((2-chloro-4- fluorophenyl)sulfonyl)phenyl)-1,3,4- thiadiazol-2-amine; |

and pharmaceutically acceptable salts thereof.

In certain instances, the compound of Formula (I) is:
N-(4-((4-chloro-3-(trifluoromethyl)phenyl)sulfonyl)phenyl)-1,3,4-thiadiazol-2-amine;
N-(4-((4-chloro-3-(trifluoromethyl)phenyl)sulfonyl)phenyl)thiazol-2-amine;
N-(5-((4-chlorophenyl)sulfonyl)pyrazin-2-yl)-5-((4-(methylsulfonyl)piperazin-1-yl)methyl)-1,3,4-thiadiazol-2-amine;
N-(4-(4-chlorophenoxyl)phenyl)-1,3,4-thiadiazol-2-amine;
N1-(4-chlorophenyl)-N4-(1,3,4-thiadiazol-2-yl)benzene-1,4-diamine; or
(4-((1,3,4-thiadiazol-2-yl)amino)phenyl)(4-chlorophenyl)methanone;
   or a pharmaceutically acceptable salt thereof.

The compounds of Formula (I) may be prepared and/or formulated as pharmaceutically acceptable salts. Pharmaceutically acceptable salts are non-toxic salts of a free base form of a compound that possesses the desired pharmacological activity of the free base. These salts may be derived from inorganic or organic acids. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of any one of Formulas (I)-(III) that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The embodiments also relate to pharmaceutically acceptable prodrugs of the compounds of any one of Formulas (I)-(III), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The embodiments also relate to pharmaceutically active metabolites of compounds of any one of Formulas (I)-(III), and uses of such metabolites in the methods of the embodiments. A “pharmaceutically active metabolite” means a pharmacologically active product of metabolism in the body of a compound of any one of Formulas (I)-(III) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Pharmaceutical Compositions

For treatment purposes, a pharmaceutical composition according to the invention comprises at least one compound of Formula (I), or a pharmaceutically acceptable salt thereof. The pharmaceutical compositions may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the embodiments are sterile compositions. Pharmaceutical compositions may be prepared using compounding techniques known or that become available to those skilled in the art.

Sterile compositions are also contemplated by the embodiments, including compositions that are in accord with national and local regulations governing such compositions.

The pharmaceutical compositions and compounds described herein may be formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the embodiments may be administered by a suitable route of delivery, such as oral, parenteral, rectal, nasal, topical, or ocular routes, or by inhalation. Preferably, the compositions are formulated for intravenous or oral administration.

For oral administration, the compounds the embodiments may be provided in a solid form, such as a tablet or capsule, or as a solution, emulsion, or suspension. To prepare the oral compositions, the compounds of the embodiments may be formulated to yield a dosage of, e.g., from about 0.01 to about 50 mg/kg daily, or from about 0.05 to about 20 mg/kg daily, or from about 0.1 to about 10 mg/kg daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The inventive compositions may be formulated for rectal administration as a suppository. For parenteral use, including intravenous, intramuscular, intraperitoneal, intranasal, or subcutaneous routes, the agents of the embodiments may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier.

For topical applications, the compounds of the present embodiments are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the embodiments may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, slowing the worsening or progression of a disease, disorder, or symptom, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

Exemplary neurodegenerative diseases that may be therapeutic targets for modulators of the PI3K-AKT-mTOR pathway include Alzheimer's Disease, Parkinson's Disease, fronto-temporal Dementia, Dementia with Lewy Bodies, PD Dementia, Multiple System Atrophy, Huntington's disease and Amyotrophic Lateral Sclerosis. In addition to neurodegenerative disorders, compounds that modulate the PI3K-AKT-mTOR pathway may also have utility in the treatment of cancer (particularly, prostate, colon, pancreatic and renal), infections, Crohn's disease, heart disease and aging.

In one aspect, the compounds and pharmaceutical compositions of the invention specifically target PI3K, AKT and/or mTOR. Thus, these compounds and pharmaceutical compositions can, by preventing, reversing, slowing, or inhibiting the P13K-AKT-mTOR pathway, treat degenerative neurological diseases related to or caused by mis-regulation of autophagy, e.g., such as inadequate clearance of protein aggregates and/or damaged organelles, insufficient activation of a survival pattern of gene expression, and/or deficiencies in cell energetics. Preferably, the methods of the invention target neurodegenerative diseases associated with the P13K-AKT-mTOR pathway. In preferred embodiments, methods of treatment target Parkinson's disease, Alzheimer's disease, Lewy body disease, multiple system atrophy, or Huntington's disease. The compounds, compositions, and method of the present invention are also used to mitigate deleterious effects that inhibit autophagy, such as impaired clearance of protein aggregates or damaged organelles. While the invention is not limited by any particular mechanism of action, dysregulation of autophagy is thought to be caused by alpha synuclein and/or beta amyloid.

In the methods of the embodiments, an “effective amount” of a PI3K-AKT-MTOR modulator means an amount sufficient to alter the phosphorylation of constituents of the PI3K-AKT-MTOR pathway, alter expression of survival genes regulated by this pathway, improve cellular energetics, increase markers of autophagy and/or decrease the accumulation of protein aggregates. Measuring one or more of these markers of modulation of the PI3I-AKT-MTOR pathway may be performed by routine analytical methods such as those described below and is useful in a variety of settings, including in vitro assays.

In treatment methods according to the embodiments, an “effective amount” means an amount or dose sufficient to generally bring about the desired therapeutic benefit in subjects needing such treatment. Effective amounts or doses of the compounds of the embodiments may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about 1 μg to 2 mg of active agent per kilogram of subject's body weight per day, preferably about 0.05 to 100 mg/kg/day, or about 1 to 35 mg/kg/day, or about 0.1 to 10 mg/kg/day. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

Drug Combinations

The inventive compounds described herein may be used in pharmaceutical compositions or methods in combination with one or more additional active ingredients in the treatment of neurodegenerative disorders. For example, additional active ingredients are those that are known or discovered to be effective in treating neurodegenerative disorders, including those active against another target associated with the disease, such as but not limited to, a) compounds that address protein misfolding (such as drugs which reduce the production of these proteins, which increase their clearance or which alter their aggregation and/or propagation); b) compounds that treat symptoms of such disorders (e.g., dopamine replacement therapies, cholinesterase inhibitors and precognitive glutamatergic drugs); and c) drugs that act as neuroprotectants by complementary mechanisms (e.g., those targeting autophagy, those that are anti-oxidants, and those acting by other mechanisms such as adenosine A2A antagonists).

For example, additional active ingredients are those that are known or discovered to be effective in treating neurodegenerative disorders, including those active against another target associated with the disease, such as but not limited to, a) compounds that target different mechanisms of protein misfolding (such as aggregation and/or propagation); b) compounds that treat symptoms of such disorders (e.g., dopamine replacement therapies); and c) drugs that act as neuroprotectants by complementary mechanisms (e.g., those targeting autophagy, anti-oxidants, and adenosine A2A antagonists).

For example, compositions and formulations of the embodiments, as well as methods of treatment, can further comprise other drugs or pharmaceuticals, e.g., other active agents useful for treating or palliative for a degenerative neurological disease related to or caused by protein aggregation, e.g., synuclein, beta-amyloid and/or tau protein aggregation, e.g., Parkinson's disease, Alzheimer's Disease (AD), Lewy body disease (LBD) and multiple system atrophy (MSA), or related symptoms or conditions. In this regard, compositions and formulations of the generic and specific compounds described herein are useful in methods of treatment for Alzheimer's Disease, Parkinson's Disease, fronto-temporal dementia, dementia with Lewy Bodies, PD dementia, multiple system atrophy, Huntington's disease, Amyotrophic lateral sclerosis, cancer, infection, Crohn's disease, heart disease, and aging. The pharmaceutical compositions of the embodiments may additional comprise one or more of such active agents, and methods of treatment may additionally comprise administering an effective amount of one or more of such active agents. In certain embodiments, additional active agents may be antibiotics (e.g., antibacterial or bacteriostatic peptides or proteins), e.g., those effective against gram positive or negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof. Additional active agents include those useful in such compositions and methods include dopamine therapy drugs, catechol-O-methyl transferase (COMT) inhibitors, monamine oxidase inhibitors, cognition enhancers (such as acetylcholinesterase inhibitors or memantine), adenosine 2A receptor antagonists, beta-secretase inhibitors, or gamma-secretase inhibitors. In particular embodiments, at least one compound of the present embodiments may be combined in a pharmaceutical composition or a method of treatment with one or more drugs selected from the group consisting of: tacrine (Cognex), donepezil (Aricept), rivastigmine (Exelon) galantamine (Reminyl), physostigmine, neostigmine, Icopezil (CP-118954, 5,7-dihydro-3-[2-[1-(phenylmethyl)-4-piperidinyl]ethyl]-6H-pyrrolo-[4,5-f-]-1,2-benzisoxazol-6-one maleate), ER-127528 (4-[(5,6-dimethoxy-2-fluoro-1-indanon)-2-yl]methyl-1-(3-fluorobenzyl)piperidine hydrochloride), zanapezil (TAK-147; 3-[1-(phenylmethyl)piperidin-4-yl]-1-(2,3,4,5-tetrahydro-1H-1-benzazepin-8-yl)-1-propane fumarate), Metrifonate (T-588; (−)—R-alpha-[[2-(dimethylamino)ethoxy]methyl]benzo[b]thiophene-5-methanol hydrochloride), FK-960 (N-(4-acetyl-1-piperazinyl)-p-fluorobenzamide-hydrate), TCH-346 (N-methyl-N-2-pyropinyldibenz[b,f]oxepine-10-methanamine), SDZ-220-581 ((S)-alpha-amino-5-(phosphonomethyl)-[1,1′-biphenyl]-3-propionic acid), memantine (Namenda/Exiba) and 1,3,3,5,5-pentamethylcyclohexan-1-amine (Neramexane), tarenflurbil (Flurizan), tramiprosate (Alzhemed), clioquinol, PBT-2 (an 8-hydroxyquinilone derivative), 1-(2-(2-Naphthyl)ethyl)-4-(3-trifluoromethylphenyl)-1,2,3,6-tetrahydropyr-idine, Huperzine A, posatirelin, leuprolide or derivatives thereof, ispronicline, (3-aminopropyl)(n-butyl)phosphinic acid (SGS-742), N-methyl-5-(3-(5-isopropoxypyridinyl))-4-penten-2-amine (ispronicline), 1-decanaminium, N-(2-hydroxy-3-sulfopropyl)-N-methyl-N-octyl-, inner salt (zt-1), salicylates, aspirin, amoxiprin, benorilate, choline magnesium salicylate, diflunisal, faislamine, methyl salicylate, magnesium salicylate, salicyl salicylate, diclofenac, aceclofenac, acemetacin, bromfenac, etodolac, indometacin, nabumetone, sulindac, tolmetin, ibuprofen, carprofen, fenbufen, fenoprofen, flurbiprofen, ketoprofen, ketorolac, loxoprofen, naproxen, tiaprofenic acid, suprofen, mefenamic acid, meclofenamic acid, phenylbutazone, azapropazone, metamizole, oxyphenbutazone, sulfinprazone, piroxicam, lornoxicam, meloxicam, tenoxicam, celecoxib, etoricoxib, lumiracoxib, parecoxib, rofecoxib, valdecoxib, nimesulide, arylalkanoic acids, 2-arylpropionic acids (profens), N-arylanthranilic acids (fenamic acids), pyrazolidine derivatives, oxicams, COX-2 inhibitors, sulphonanilides, essential fatty acids, and Minozac (2-(4-(4-methyl-6-phenylpyridazin-3-yl)piperazin-1-yl)pyrimidine dihydrochloride hydrate). Such a combination may serve to increase efficacy, ameliorate other disease symptoms, decrease one or more side effects, or decrease the required dose of an inventive compound. The additional active ingredients may be administered in a separate pharmaceutical composition from a compound of the embodiments or may be included with a compound of the embodiments in a single pharmaceutical composition. The additional active ingredients may be administered simultaneously with, prior to, or after administration of a compound of Formula (I).

Chemical Synthesis

The embodiments are also directed to processes and intermediates useful for preparing subject compounds or a salt or solvate or stereoisomer thereof.

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001.)

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd ed., ed. L. R. Snyder and J. J. Kirkland, John Wiley and Sons, 1979; and Thin Layer Chromatography, E. Stahl (ed.), Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as T. W. Greene and P. G. M. Wuts, “Protective Groups in Organic Synthesis,” 4^{th} ed., Wiley, New York 2006. The protecting groups may be removed at a convenient subsequent stage using methods known from the art.

Exemplary chemical entities useful in methods of the embodiments will now be described by reference to illustrative synthetic schemes for their general preparation herein and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups. Each of the reactions depicted in the general schemes is preferably run at a temperature from about 0° C. to the reflux temperature of the organic solvent used. Unless otherwise specified, the variables are as defined above in reference to Formula (I). Isotopically labeled compounds as described herein are prepared according to the methods described below, using suitably labeled starting materials. Such materials are generally available from commercial suppliers of radiolabeled chemical reagents.

Representative syntheses for compounds of Formula (I) are described in Schemes 1-23, and the particular examples that follow.

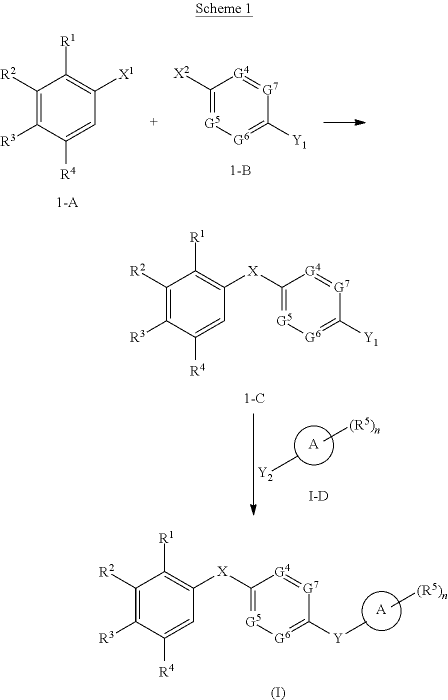

In Scheme 1, R¹, R², R³, R⁴, R⁵, X, G⁴, G⁵, G⁶, G⁷, Ring A, Y, and n are as defined herein. As discussed below, X¹ and X² comprise a nucleophile and a leaving group and Y¹ is a precursor moiety to forming a linkage Y in Formula (I). Starting materials may be obtained from commercial sources or via well-established synthetic procedures.

Referring to Scheme 1, arylation of Compound 1-A with Compound 1-B through a nucleophilic reaction forms Compound 1-C. In Compound 1-A and 1-B, one of X¹ and X² comprises a nucleophile and the other comprises a leaving group. The nucleophile is selected to provide the appropriate functional group in the X substituent. X¹ and/or X² can comprise an alkylene group. In certain instances, when X comprises O, X¹ or X² can comprise an alcohol. In certain instances, when X comprises S or SO₂, X¹ or X² can comprise a thiol or a sulfonyl halide. In certain instances, when X comprises NR^{a}, X¹ or X² can comprise an amino group. The nucleophile can react in a nucleophilic aromatic substitution in which the nucleophile displaces a leaving group on the other reactant. Examples of leaving groups include, but are not limited to, halogen, triflate, fluorosulfonate, tosylate, or mesylate. In certain instances, a Friedel Crafts acylation reaction between Compound 1-A and 1-B can be used when X comprises CO. One of skill in the art will recognize that couplings can be performed that yield a product with a lower oxidation state than that desired, such as a sulfide or secondary hydroxyl group, which can then be oxidized using methods known in the art to the corresponding sulfone or ketone.

With continued reference to Scheme 1, Compound 1-C is converted to a compound of Formula (I). In Scheme 1, Y¹ comprises a precursor moiety to form linkage Y in Compound (I). Y¹ can comprise an alkylene group.

For example, Y¹ can be a functional group or be converted to a functional group that can readily react in a cyclization reaction to form linkage Y and Ring A. In certain instances, Y¹ comprises an amino group, which can be converted to a thiocyanate group. The thiocyanate group can serve as a chemical handle to form linker Y and Ring A.

In other examples, Y¹ can be a functional group or be converted to a functional group that can readily react in a nucleophilic reaction to form linkage Y and Ring A. In certain instances, Y¹ comprises an amino group, which can react with an activated acyl group or sulfonyl group attached to Ring A. In certain instances, Y¹ comprises an amino group, which can react in a nucleophilic substitution reaction with a leaving group on Ring A to displace the leaving group and directly attach to Ring A. In such instances, Ring A is prepared prior to the coupling that forms linkage Y. General methods for preparing unsubstituted and substituted 5-membered heteroaryl rings are well known in the art (see, for example, Katritzky et al., Handbook of Heterocyclic Chemistry, 3^{rd} ed., 2010, Chapter 4). In certain instances, Y¹ comprises a thiol or hydroxyl group, which can react in a nucleophilic reaction to form a compound, where Y is S or O. Alternatively, the thiol group can be oxidized to a sulfonyl or sulfonamide group for further reaction to introduce Ring A.

In certain instances, Y¹ comprises an activated acyl group, which can serve as a chemical handle to form linker Y and Ring A. An acyl group with a leaving group can react with various reagents to form a compound where Y is —CO—, —CO₂—, —CONH—. For example, an acyl halide can react in a Freidel Crafts acylation manner to introduce Ring A. An acyl halide can react with an alcohol to form an ester or with an amine to form an amide group. The alcohol or amino groups can already be attached to Ring A or the ester or amide groups can further react to introduce Ring A.

Accordingly and as described in more detail herein, the present disclosure provides a process of preparing a compound of the present disclosure, the process involves:

reacting a compound of formula:

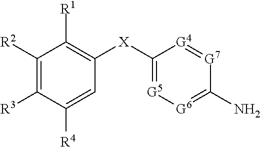

with

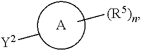

thereby producing a compound of Formula (I):

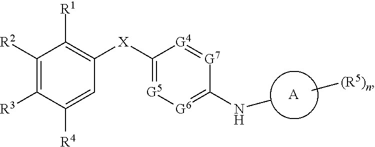

wherein R¹, R², R³, R⁴, R⁵, n, X, G⁴, G⁵, G⁶, G⁷, Ring A, and Y² are as described herein.

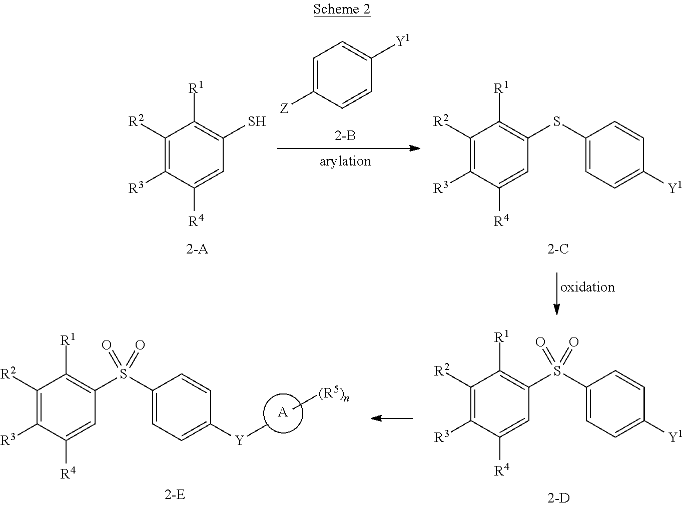

In Scheme 2, R¹, R², R³, R⁴, R⁵, Ring A, Y, and n are as defined herein. As discussed below, Z is a leaving group and Y¹ is a precursor moiety to forming a linkage Y in Compound 2-E. Starting materials may be obtained from commercial sources or via well-established synthetic procedures.

Referring to Scheme 2, arylation of Compound 2-A with Compound 2-B through a nucleophilic reaction forms Compound 2-C. In Compound 2-B, Z is a leaving group, such as a halogen, triflate, fluorosulfonate, tosylate, or mesylate. The thiol group of Compound 2-C can be oxidized to a sulfone group (or to a sulfoxide, not shown) to form Compound 2-D. Suitable oxidizing reagents for oxidation of the thiol group include, but are not limited to, hydrogen peroxide and mCPBA.

With continued reference to Scheme 2, Compound 2-D is converted to Compound 2-E. In Scheme 2, Y¹ is a precursor moiety to forming linkage Y in Compound 2-E. For example, Y¹ can be an amino group.

Scheme 3 shows synthetic schemes where intermediate compounds comprise Y¹ as an amino group and are reacted to form compounds comprising thiadiazole and thiazole rings as the A ring.

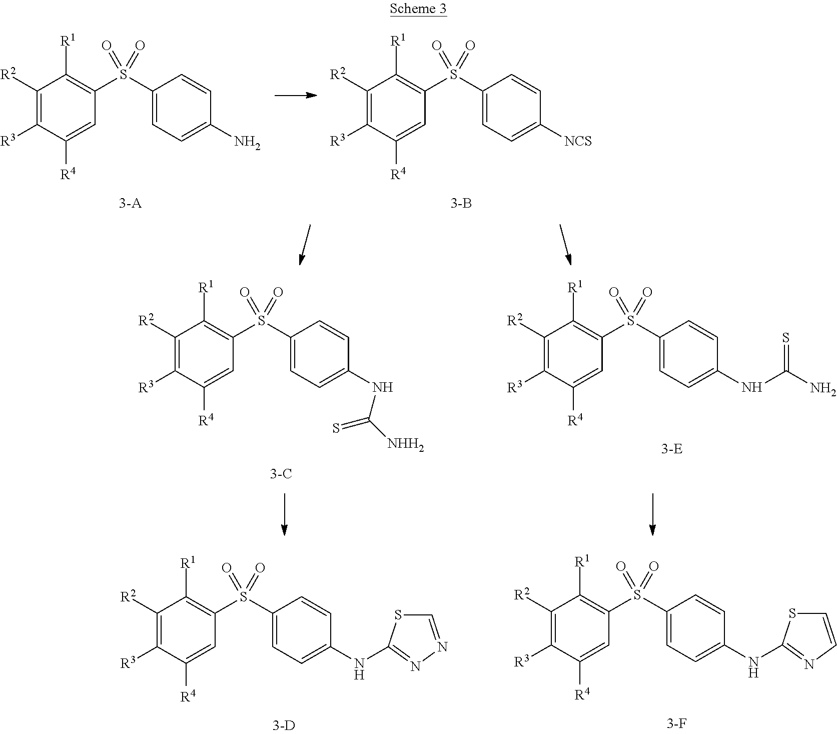

Referring to Scheme 3, the amino group of Compound 3-A is converted to an isothiocyanate, which can serve as a chemical handle to form linker Y and Ring A. Compound 3-B reacts with hydrazine to form Compound 3-C with a hydrazinecarbothioamide group. Compound 3-C can undergo a cyclization reaction with a formyl synthon to form Compound 3-D. Examples of compounds that can be used as a formyl synthon for the reaction include triethoxymethane and trimethoxymethane.

With continued reference to Scheme 3, Compound 3-B reacts with ammonia to form Compound 3-E with a thiourea group. The thiourea group can undergo Hantzche thiazole reaction to form Compound 3-F. The Hantzche thiazole reaction uses a haloketone or haloketone equivalent or to react with a thiourea. A haloketone equivalent can be a haloketone that is protected at the ketone moiety. In certain instances, the haloketone or haloketone equivalent is 2-bromo-1,1-dimethoxyethane. One of skill in the art will recognize that the transformations depicted in Scheme 3 may also be used to generate compounds of Formula (I) in which X is a group other than —SO₂— and in which the central aromatic ring is other than phenyl.

Accordingly and as described in more detail herein, the present disclosure provides a process of preparing a compound of the present disclosure, the process involves:

reacting a compound of formula:

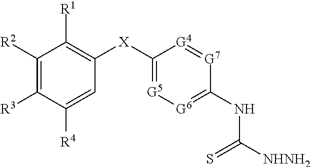

with a formyl synthon,
thereby producing a compound of formula:

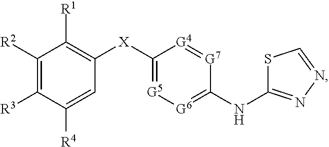

wherein R¹, R², R³, R⁴, X, G⁴, G⁵, G⁶, G⁷ are described herein.

In certain instance, the formyl synthon is triethoxymethane.

Accordingly and as described in more detail herein, the present disclosure provides a process of preparing a compound of the present disclosure, the process involves:

reacting a compound of formula:

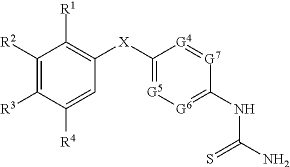

with a haloketone or haloketone equivalent,
thereby producing a compound of formula:

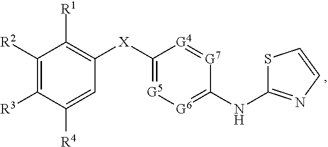

wherein R¹, R², R³, R⁴, X, G⁴, G⁵, G⁶, G⁷ are described herein.

In certain instances, the haloketone or haloketone equivalent is 2-bromo-1,1-dimethoxyethane.

In certain instances, the above processes further involving the step of forming a salt of a compound of the present disclosure. Embodiments are directed to the other processes described herein; and to the product prepared by any of the processes described herein.

In certain instances, the above processes further involving the step of forming a salt of a compound of the present disclosure. Embodiments are directed to the other processes described herein; and to the product prepared by any of the processes described herein.

### EXAMPLES

The following examples are offered to illustrate but not to limit the invention.

### Example 1

### N-(4-((4-Chloro-3-(trifluoromethyl)phenyl) sulfonyl)phenyl)-1,3,4-thiadiazol-2-amine

The title compound was prepared as shown in Scheme 4.

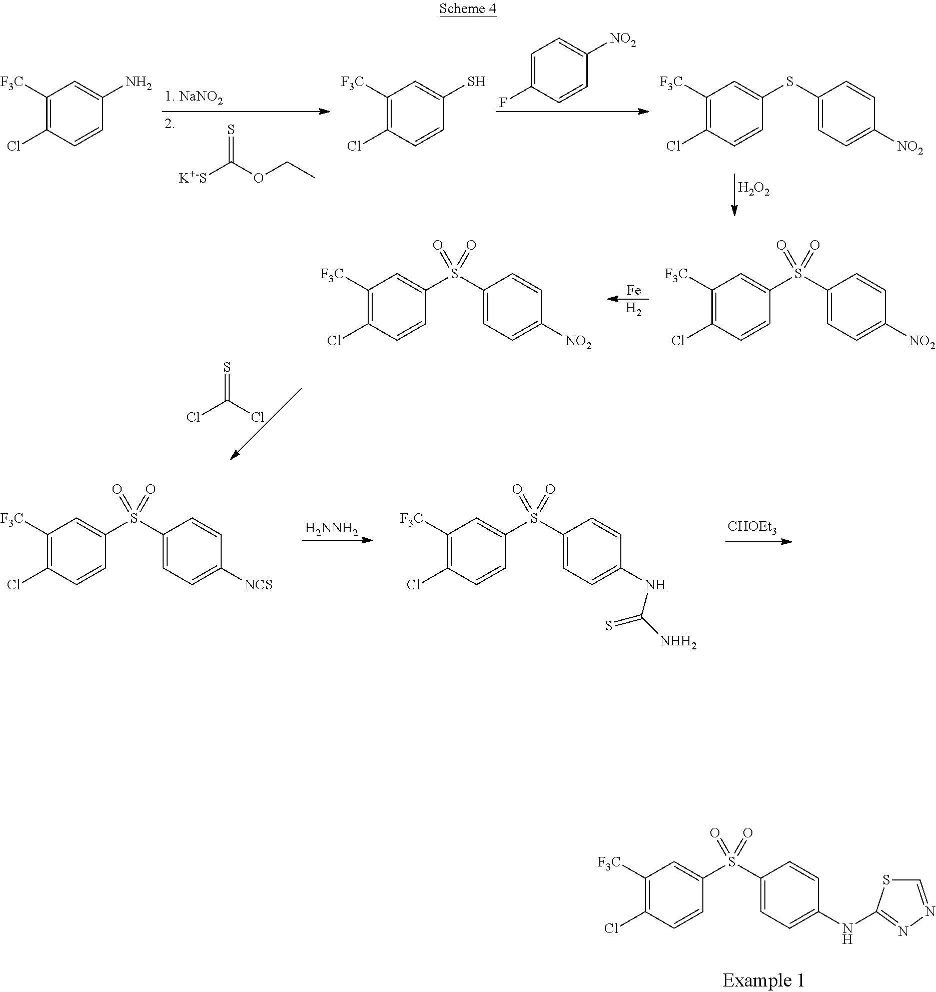

Step 1. In a 1 L round-bottom flask equipped with mechanical stirrer and thermometer was added 60 mL of concentrated hydrochloric acid, 60 mL of water, and 4-chloro-3-(trifluoromethyl)benzene amine (19.5 g, 0.1 mol). The mixture was heated to promote dissolution and then cooled down to below 0° C. in ice-water bath. A solution of sodium nitrite (7.6 g, 0.11 mol) in 10 mL of water was added in dropwise while the internal temperature was kept below 5° C. and the mixture was stirred at 5° C. for 30 min. The mixture was then added into a mixture of potassium ethyl xanthate (19.2 g, 0.12 mol) in 30 mL of water over 2 hours. Upon the completion of reaction (about 30 min), the organic phase in reaction mixture was separated, and the aqueous layer was extracted twice with diethyl ether. The combined organic layers were washed with 30 mL of 10% sodium hydroxide solution followed by several portions of water until the aqueous phase that separated was pH neutral. The organic phase was dried over Na₂SO₄ and concentrated and the crude residue was dissolved in 95% ethanol (100 mL). The solution heated to reflux to aid dissolution. To this hot solution was added potassium hydroxide pellets (23.5 g, 0.42 mol) slowly so that the solution kept gentle refluxing until all the material was completely dissolved in water (about 8 hours). Approximately 80 mL of ethanol was then removed by distillation on a steam bath, and the residue was taken up in the minimum of water (about 100 mL). The aqueous solution was extracted with diethyl ether (50 mL x 3). The pH of aqueous layer was adjusted to 1 with 6 N sulfuric acid. Extraction with diethyl ether (50 mL x 3) was performed, and the combined organic layers were dried over Na₂SO₄ and concentrated to give the crude product, which was purified by column chromatography (0 to 2% ethyl acetate/petroleum ether) to give 4-chloro-3-(trifluoromethyl)benzenethiol (16.1 g, 75%) as yellow solid.

Step 2. To a solution of 4-chloro-3-(trifluoromethyl)benzenethiol (19.2 g, 0.091 mol) in N,N-dimethylformamide (250 mL) was added 1-fluoro-4-nitrobenzene (12.8 g, 0.091 mol) and Cs₂CO₃ (59.4 g, 0.182 mol) and the reaction mixture was stirred at 80° C. under thin layer chromatography monitoring (1:30 ethyl acetate/petroleum ether). Upon the completion of the reaction, the mixture was cooled to room temperature and diluted with water (500 mL). The aqueous layer was extracted with ethyl acetate (200 mL x 3) and the combined organic layers were washed with brine, dried over Na₂SO₄, filtered and concentrated to give crude 4-chloro-3-(trifluoromethyl)phenyl)(4-nitrophenyl)sulfane (25 g, 82%) as a yellow oil, which was used in the next step without further purification.

Step 3. To a solution of 4-chloro-3-(trifluoromethyl)phenyl)(4-nitrophenyl)sulfane (25 g, 0.075 mol) in acetic acid (100 mL) was added 30% H₂O₂ dropwise (20 g, 0.3 mol) at room temperature. Then the reaction mixture was stirred at 85° C. with thin layer chromatography monitoring (1:5 ethyl acetate/petroleum ether). Upon the completion of reaction, water was added to quench the reaction. The aqueous layer was extracted with ethyl acetate (100 mL x 3) and the combined organic layers were washed with brine, dried over Na₂SO₄, filtered and concentrated to give the crude product, which was purified by flash chromatography (0 to 10% ethyl acetate/petroleum ether) to give 1-chloro-4-(4-nitrophenylsulfonyl)-2-(trifluoromethyl)benzene (20.8 g, 76%) as a white solid.

Step 4. Five drops of concentrated HCl was added into a mixture of iron power (16 g, 0.29 mol) in water (100 mL) and ethanol (100 mL). The mixture was heated to reflux while 1-chloro-4-(4-nitrophenylsulfonyl)-2-(trifluoromethyl)benzene (26.4 g, 0.072 mol) was added. The reaction mixture was kept refluxing for additional 1 hour with thin layer chromatography monitoring (1:5 ethyl acetate/petroleum ether). Upon the completion of reaction, the hot mixture was filtered and the filter cake was washed with ethanol. The pH of filtrate was adjusted to 10 with 2 N NaOH and the water phase was extracted with ethyl acetate (100 mL x 3). The combined organic layers were washed with brine, dried over Na₂SO₄, filtered and concentrated to give the crude product, which was purified by flash chromatography (0 to 15% ethyl acetate/petroleum ether) to give 4-((4-chloro-3-(trifluoromethyl)phenyl)sulfonyl) aniline as a white solid (19.4 g, 79%).

Step 5. Thiophosgene (6.6 g, 0.057 mol) was added into a two phase solution of 4-((4-chloro-3-(trifluoromethyl)phenyl)sulfonyl) aniline (19.2 g, 0.057 mol) in dichloromethane and water containing sodium bicarbonate (13.4 g, 0.13 mol) at 0° C. The reaction mixture was stirred at 0° C. for 2 hours. Upon the completion of reaction, the organic layer was separated, dried over Na₂SO₄, filtered and concentrated to dryness. The residue was purified by column chromatography (0 to 50% ethyl acetate/petroleum ether) to give 1-chloro-4-(4-isothiocyanatophenylsulfonyl)-2-(trifluoromethyl)benzene (11.5 g, 53%) as a yellow solid.

Step 6. Hydrazine monohydrate (5.2 g, 0.058 mol) was added into a solution of 1-chloro-4-(4-isothiocyanatophenylsulfonyl)-2-(trifluoromethyl)benzene (11 g, 0.029 mol) in ethanol (60 mL) dropwise at 0° C. After 4 hours, the reaction mixture was diluted with water (100 mL) and extracted with dichloromethane (50 mL x 3). The combined organic layers were washed with brine, dried over Na₂SO₄, filtered and concentrated to give crude N-(4-((4-chloro-3-(trifluoromethyl)phenyl)sulfonyl)phenyl)hydrazinecarbothioamide (8.4 g, 70%), which was used in the next step without further purification.

Step 7. N-(4-((4-Chloro-3-(trifluoromethyl)phenyl)sulfonyl)phenyl)hydrazinecarbothioamide (8.2 g, 0.02 mol) was treated with triethoxymethane (50 mL) at 145° C. for 3 hours. Water (100 mL) was added and the mixture was extracted with dichloromethane (50 mL x 3). The combined organic layers were washed with brine, dried over Na₂SO₄, filtered and concentrated to give the crude product, which was purified by column chromatography (0 to 10% ethyl acetate/petroleum ether) to give the title compound (5.4 g, 64%) as a white solid. ¹H NMR (300 MHz, DMSO-d₆) δ 14.12 (s, 1H), 8.81 (s, 1H), 8.42-8.31 (m, 4H), 8.08-8.04 (m, 3H). LCMS ES+ (m/z), 419.9 and 421.9 (M+1)+, Cl pattern found.

### Example 2

### N-(4-((4-Chloro-3-(trifluoromethyl)phenyl)sulfonyl)phenyl)-1,3,4-thiadiazol-2-amine

The title compound was prepared as shown in Scheme 5.

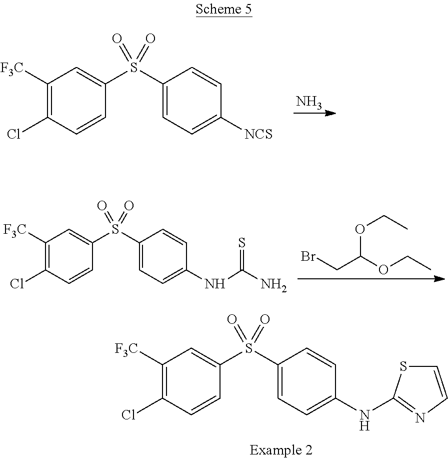

Step 1. An ammonia/ethanol solution (equal to 16 mmol of ammonia) was added into a solution of 1-chloro-4-(4-isothiocyanatophenylsulfonyl)-2-(trifluoromethyl)benzene (2.0 g, 5.3 mmol) in ethanol (10.0 mL). The reaction mixture was heated at 50° C. with thin layer chromatography monitoring. Upon the completion of reaction, the reaction mixture was concentrated to afford crude 1-(4-(4-chloro-3-(trifluoromethyl)phenylsulfonyl)phenyl)thiourea (2.08 g, quant.) as a yellow solid, which was used in the next step without further purification.

Step 2. 2-Bromo-1,1-dimethoxyethane (1.1 g, 6.1 mmol) was added into a solution of crude 1-(4-(4-chloro-3-(trifluoromethyl)phenylsulfonyl)phenyl)thiourea (2.0 g, 5.1 mmol) in acetic acid (10 mL). The reaction mixture was heated to reflux for 2 hours with thin layer chromatography monitoring. After completion of reaction, the reaction mixture was concentrated and the residue was taken up with dichloromethane, washed with saturated aqueous sodium bicarbonate and brine successively, dried and concentrated to give the crude product, which was purified by column chromatography (0 to 10% ethyl acetate/petroleum ether) to give the title compound (1.02 g, 48%) as an off-white solid. ¹H NMR (300 MHz, DMSO-d₆) δ 10.87 (s, 1H), 8.26-8.03 (m, 2H), 8.03-8.01 (m, 3H), 8.01-7.99 (m, 2H), 7.38 (s, 1H), 7.12 (d, 2H). LCMS ES+ (m/z), 419.0 and 421.0 (M+1)+, Cl pattern found.

### Example 3

### N-(4-((4-Chlorophenyl)sulfonyl)phenyl)-5-((4-(methylsulfonyl)piperazin-1-yl)methyl)-1,3,4-thiadiazol-2-amine

The title compound was prepared as shown in Scheme 6.

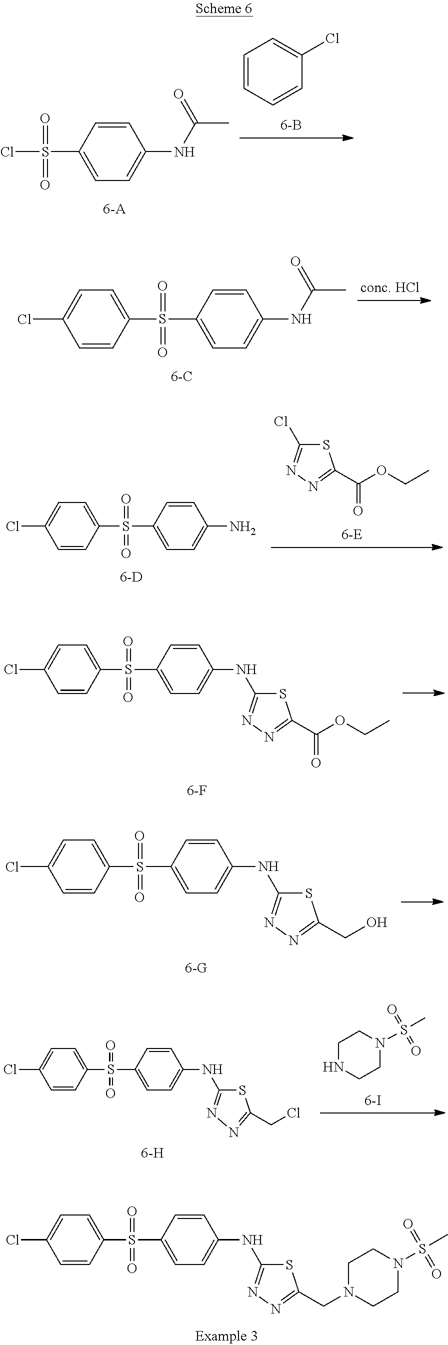

Step 1. A mixture of Compound 6-A (5 g, 0.02 mol) and Compound 6-B (20 mL) was warmed to 50° C., and AlCl₃ (5.7 g, 0.042 mol) was added in portions while maintaining temperature below 65° C. of the reaction mixture. The resulting mixture was stirred for 3 hours at about 70-80° C. When thin layer chromatography monitoring (petrol ether:ethyl acetate=2:1 and R_{f} at 0.5) showed the reaction was complete, the mixture was poured into ice-water (50 mL) and 4 N HCl (50 mL) and extracted with CH₂Cl₂ (50 mL x 3). The combined organic layers were dried over Na₂SO₄ and concentrated to give Compound 6-C (5.5 g, 89%) as a light brown solid. ¹H NMR (400 MHz DMSO) δ 2.10 (s, 3H), 7.42-7.44 (d, J=8.8 Hz, 2H), 7.69-7.71 (d, J=9.2 Hz, 2H), 7.77-7.84 (m, 4H), 9.75 (s, 1H).

Step 2. A mixture of Compound 6-C (5.5 g, 0.014 mol) in concentrated HCl (70 mL) and ethanol (70 mL) was refluxed overnight. Monitoring by thin layer chromatography (petrol ether:ethyl acetate=2:1 and R_{f} at 0.3) showed the reaction was complete. The mixture was concentrated. The residue was neutralized with saturated NaHCO₃ to pH >7 and extracted with ethyl acetate (80 mL x 3). The combined organic layers were dried and concentrated to give Compound 6-D (4.1 g, 87%) as a light brown solid.

Step 3. A mixture of Compound 6-D (4.1 g, 0.015 mol), Compound 6-E (2.33 g, 0.013 mol) and p-toluenesulfonic acid (2.85 g, 0.015 mol) in ethanol (70 mL) was refluxed overnight. Monitoring by thin layer chromatography (petrol ether:ethyl acetate=2:1 and R_{f} at 0.5) showed the reaction was complete. The mixture was partitioned between saturated aqueous NaHCO₃ (100 mL) and ethyl acetate (100 mL) and extracted with ethyl acetate (100 mL x 2). The combined organic layers were dried and concentrated. The residue was recrystallized with ethyl acetate to give Compound 6-F (2.8 g, 44%) as light brown solid. ¹H NMR (400 MHz, MeOH) δ 1.37-1.41 (m, 3H), 4.40-4.48 (m, 2H), 7.42-7.47 (m, 2H), 7.78-7.87 (m, 4H).

Step 4. To a solution of Compound 6-F (2.2 g, 0.005 mol) in tetrahydrofuran (60 mL) at 0° C. was added LiAlH₄ (0.6 g, 0.015 mol) in portions. The resulting mixture was stirred for 1 hour at room temperature. Monitoring by thin layer chromatography (petrol ether:ethyl acetate=1:1 and R_{f} at 0.6) showed the reaction was complete. The mixture was quenched with water (0.6 mL) and 10% NaOH (0.6 mL) and then filtered. The filter cake was washed with tetrahydrofuran (200 mL) and the filtrate was concentrated. The residue was recrystallized with methyl tert-butyl ether (MTBE) to give Compound 6-G (1.65 g, 83%) as a yellow solid.

Step 5. A mixture of Compound 6-G (0.9 g, 4.7 mmol) in SOCl₂ (20 mL) was stirred for 2 hours at 40° C. Monitoring by thin layer chromatography (petrol ether:ethyl acetate=1:1 and R_{f} at 0.3) showed the reaction was complete. The mixture was quenched with saturated aqueous NaHCO₃ to pH >7 and extracted with ethyl acetate (50 mL x 3). The combined organic layers were dried and concentrated. The residue was washed with MTBE to give Compound 6-H (0.8 g, 85%) as a light brown solid.

Step 6. To a solution of Compound 6-H (0.8 g, 2 mmol) and Compound 6-I (0.33 g, 2 mmol) in dimethylformamide (15 mL) was added triethylamine (0.56 mL, 4 mmol) at 0° C. The resulting mixture was stirred for 1 hour at 25° C. Monitoring by thin layer chromatography (petrol ether:ethyl acetate=1:1 and R_{f} at 0.6) showed the reaction was complete. The mixture was partitioned between water (20 mL) and ethyl acetate (20 mL) and extracted with ethyl acetate (20 mL x 3). The combined organic layers were dried and concentrated. The residue was purified by preparative HPLC to give the title compound (260 mg, 24%) as an off-white solid. ¹H NMR (400 MHz, DMSO) δ 2.56 (br, 4H), 2.86 (s, 3H), 3.10 (br, 4H), 3.86 (s, 2H), 7.65-7.67 (d, J=8.8 Hz, 2H), 7.78-7.80 (d, J=8.8 Hz, 2H), 7.90-7.92 (m, 4H), 10.88 (s, 1H). MS: 528.1 (M+1⁺).

### Example 4

### N-(4-(4-Chlorophenoxyl)phenyl)-1,3,4-thiadiazol-2-amine

The title compound was prepared as shown in Scheme 7.

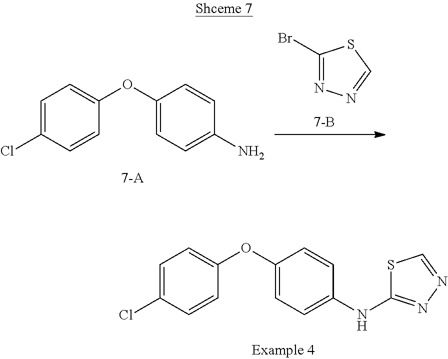 [SMILES: Nc1ccc(Oc2ccc(Cl)cc2)cc1 ; Brc1nncs1 ; Clc1ccc(Oc2ccc(Nc3nncs3)cc2)cc1]

To a solution of Compound 7-A (1.35 g, 6.2 mmol) and compound 7-B (0.4 g, 2.4 mmol) in isopropanol (25 mL) was added p-toluenesulfonic acid (1.16 g, 6.1 mmol). The resulting mixture was stirred overnight at about 60-80° C. overnight under nitrogen. Monitoring by thin layer chromatography (petrol ether:ethyl acetate=2:1 and R_{f} at 0.5) showed the reaction was complete. The mixture was diluted with saturated aqueous NaHCO₃ (90 mL) and extracted with ethyl acetate (30 mL x 3). The organic layers were concentrated and purified by silica gel column (petrol ether:ethyl acetate=15:1) to give the title compound (206 mg, 28%) as a solid. ¹H NMR (400 MHz, DMSO) δ 6.97-6.95 (d, J=9.2 Hz, 1H), 7.06-7.04 (d, J=8.4 Hz, 1H), 7.39-7.37 (d, J=8.4 Hz, 1H), 7.67-7.65 (d, J=9.2 Hz, 1H), 8.87 (s, 1H), 10.39 (s, 1H). MS: 303.9 (M+1⁺).

### Example 5

### N¹-(4-Chlorophenyl)-N⁴-(1,3,4-thiadiazol-2-yl)benzene-1,4-diamine

The title compound was prepared as shown in Scheme 8.

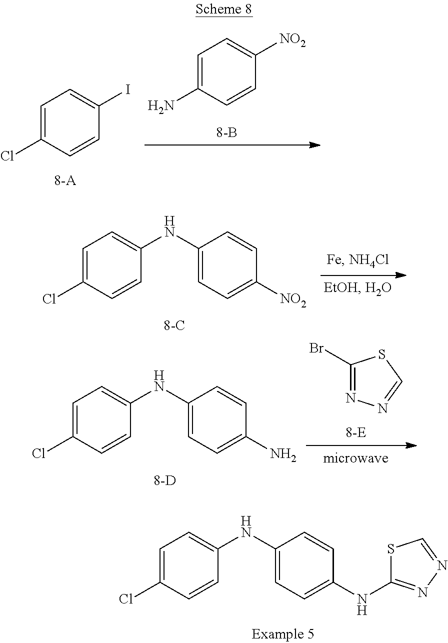

Step 1. To a mixture of Compound 8-A (0.5 g, 2.1 mmol), Compound 8-B (0.35 g, 2.5 mmol), K₃PO₄ (1.3 g, 6.3 mmol) and 2,2′-bis(diphenylphosphino)-1,1′-binaphthyl (BINAP) (0.07 g, 0.1 mmol) in toluene (5 mL) was added Pd₂(dba)₃ (tris(dibenzylideneacetone)dipalladium(0)) (0.06 g, 0.1 mmol) The mixture was stirred at about 80-100° C. overnight under nitrogen atmosphere. Monitoring by thin layer chromatography (petrol ether:ethyl acetate=2:1 and R_{f} at 0.5) showed the reaction was complete. The mixture was washed with water and extracted with ethyl acetate (10 mL x 3). The combined organic layers were dried over Na₂SO₄ and concentrated. The residue was purified by silica gel column (petrol ether:EtOAc=100:1-10:1) to give Compound 8-C (0.12 g, 23%) as a yellow solid. ¹H NMR (400 MHz, DMSO) δ 7.09-7.11 (d, J=8.4 Hz, 2H), 7.25-7.27 (d, J=8.4 Hz, 2H), 7.37-7.39 (d, J=8.8 Hz, 2H), 8.05-8.07 (d, J=9.2 Hz, 2H), 9.70-9.77 (m, 1H), 10.39 (s, 1H).

Step 2. To a mixture of Compound 8-C (4.5 g, 18.1 mmol) and iron powder (1.52, 27.2 mmol) in ethanol/water (50 ml/100 ml) was added NH₄Cl (3.9 g, 72.6 mmol). The mixture was stirred at 50° C. for 3 hours. Monitoring by thin layer chromatography (petrol ether:ethyl acetate=1:1 and R_{f} at 0.3) showed the reaction was complete. The mixture was filtered through diatomaceous earth, and the filter cake was washed with ethanol. The filtrate was concentrated to give the crude Compound 8-D which was purified by chromatography on silica gel (petrol ether:ethyl acetate=10:1-2:3) to give Compound 8-D (3.0 g, 76%) as black oil. ¹H NMR (400 MHz, DMSO) δ 4.79 (s, 2H), 6.54-6.51 (d, J=8.8 Hz, 2H), 6.71-6.73 (d, J=8.8 Hz, 2H), 6.78-6.80 (d, J=8.4 Hz, 2H), 7.60 (s, 1H).

Step 3. To a solution of Compound 8-D (0.4 g, 1.8 mmol) and Compound 8-E (0.31 g, 1.8 mmol) in isopropanol (8 mL) was added p-toluenesulfonic acid (0.36 g, 1.9 mmol). The mixture was sealed and heated in microwave at 120° C. for 1 hour. Monitoring by thin layer chromatography (petrol ether:ethyl acetate=1:1 and R_{f} at 0.6) showed that Compound 8-E was consumed. The mixture was diluted with saturated aqueous NaHCO₃ (20 mL) and extracted with ethyl acetate (15 mL x 3). The organic layers were concentrated and the residue was purified by preparative HPLC to give the title compound (38 mg, 1.3%, combined with other 5 batches; purity 79% by HPLC) as a white solid. ¹H NMR (400 MHz, DMSO) δ 6.94-6.96 (d, J=8.4 Hz, 1H), 7.07-7.05 (d, J=8.4 Hz, 1H), 7.17-7.19 (d, J=8.8 Hz, 1H), 7.50-7.52 (d, J=8.4 Hz, 1H), 8.14 (s, 1H), 8.80 (s, 1H), 10.19 (s, 1H). MS: 302.9 (M+1⁺).

### Example 6

### (4-((1,3,4-Thiadiazol-2-yl)amino)phenyl)(4-chlorophenyl)methanone

The title compound was prepared as shown in Scheme 9.

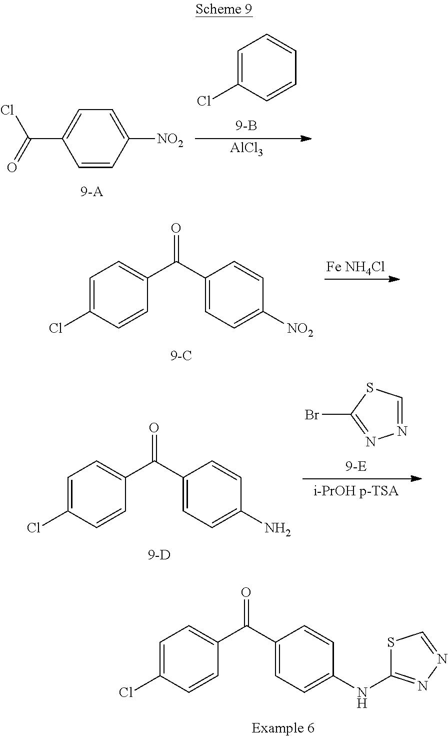

Step 1. To a solution of Compound 9-A (5.55 g, 0.03 mol) in Compound 9-B (6.72 g, 0.06 mol) was added AlCl₃ (7.61 g, 0.06 mol) in one portion at room temperature, then the mixture was refluxing overnight. Monitoring by thin layer chromatography (petrol ether:ethyl acetate=4:1 and R_{f} at 0.7) showed the reaction was complete. The mixture was poured into ice water (50 mL) and extracted with ethyl acetate (50 mL x 2). The organic layer was washed with brine, dried over Na₂SO₄ and concentrated to give Compound 9-C (3.3 g, 40%) as a dark solid. ¹H NMR (400 MHz, DMSO) δ 8.36-8.34 (m, 2H), 7.95-7.93 (m, 2H), 7.78-7.76 (m, 2H), 7.65-7.63 (m, 2H).

Step 2. To a solution of Compound 9-C (1.1 g, 4.2 mmol) in ethanol/water (10 mL/20 ml) was added iron powder (2.4 g, 42 mmol) and NH₄Cl (1.4 g, 26 mmol). Then the mixture was stirred for 3 hour at 60° C. Monitoring by thin layer chromatography (petrol ether:ethyl acetate=4:1 and R_{f} at 0.5) showed the reaction was complete. The mixture was cooled to room temperature and then filtered. The filtrate was concentrated to give Compound 9-D (0.92 g, 95%) as a yellow solid.

Step 3. To a solution of Compound 9-D (0.2 g, 0.86 mmol) in isopropanol (9 mL) was added Compound 9-E (0.14 g, 0.86 mmol) and p-toluenesulfonic acid (0.16 g, 0.86 mmol). Then the mixture was refluxed overnight. Monitoring by thin layer chromatography (petroleum ether/ethyl acetate=4:1 and R_{f} at 0.2) showed the reaction was almost complete. The solvent was removed. The residue was purified by silica gel chromatography and preparative HPLC to give the title compound (181 mg, combined with other 2 batches, yield=18%) as a yellow solid. ¹H NMR (400 MHz, DMSO) δ 8.64 (s, J=8 Hz, 1H), 7.66-7.61 (m, 4H), 7.57-7.53 (m, 4H). MS: 316.0 (M+1⁺).

### Example 7

### N-(4-(4-Chloro-3-(trifluoromethyl)phenoxy)phenyl)-1,3,4-thiadiazol-2-amine

The title compound may be prepared as shown in Scheme 10.

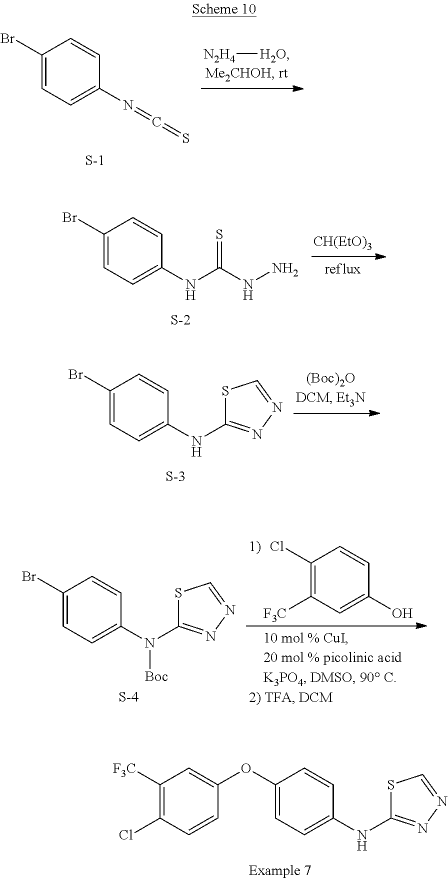

Referring to Scheme 10, compound S-4 is commercially available or is prepared as shown above. (Huang, H. et al., J. Med. Chem, 2010, 53 (8), 3048-3064. Stanovic, B.; Tisler, M., J. Org. Chem., 1960, 25 (12), 2234-2236.) Compounds S-1 and S-2 can be synthesized in a manner similar to the intermediates used for the synthesis of Example 1. The amine of Compound S-3 can be N-Boc protected. The copper mediated diaryl ether formation, to give the title compound, can be accomplished as described by Buchwald and co-workers. (Maiti, D.; Buchwald, S. L., J. Org. Chem., 2010, 75, 1791-1794.)

### Example 8

### N¹-(4-Chloro-3-(trifluoromethyl)phenyl)-N⁴-(1,3,4-thiadiazol-2-yl)benzene-1,4-diamine

The title compound may be prepared from Compound S-4 as shown in Scheme 11.

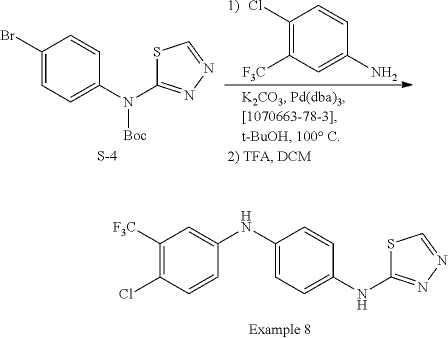

Referring to Scheme 11, for the synthesis of Example 8, a palladium mediated coupling with BretPhos as a ligand can afford the amine-linked analog. (Tsvelikhovsky, D.; Buchwald, S. L., J. Am. Chem. Soc., 2011, 133 (36), 14228-14231.)

Alternate syntheses for Compounds 7 and 8 are shown in Scheme 12. Instead of Compound S-4 as the common intermediate, Compound S-9 may be used as the common intermediate. (Yang, S.-W. et al., Bioorg. Med. Chem. Lett., 2012, 22(1), 235-239.; Letavic, M. A. et al., Bioorg. Med. Chem. Lett. 2007, 17(17), 4799-4803; Ackerman, L. et al., Org. Lett., 2011, 13, (7), 1784-1786.)

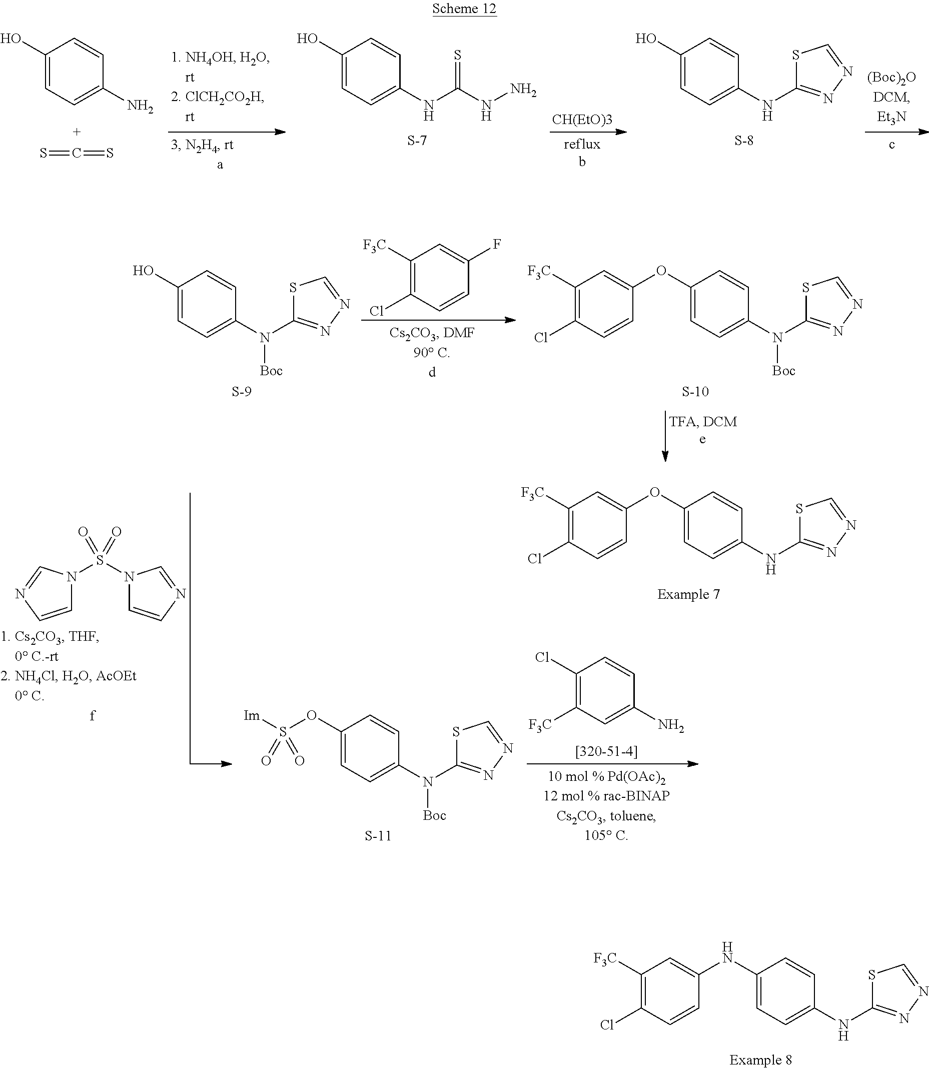

### Example 9

### N-(4-(4-Chloro-3-(trifluoromethyl)benzyl)phenyl)-1,3,4-thiadiazol-2-amine and

### Example 10

### (4-((1,3,4-Thiadiazol-2-yl)amino)phenyl)(4-chloro-3-(trifluoromethyl)phenyl)methanone

Compounds 9 and 10 may be prepared as shown in Scheme 13.

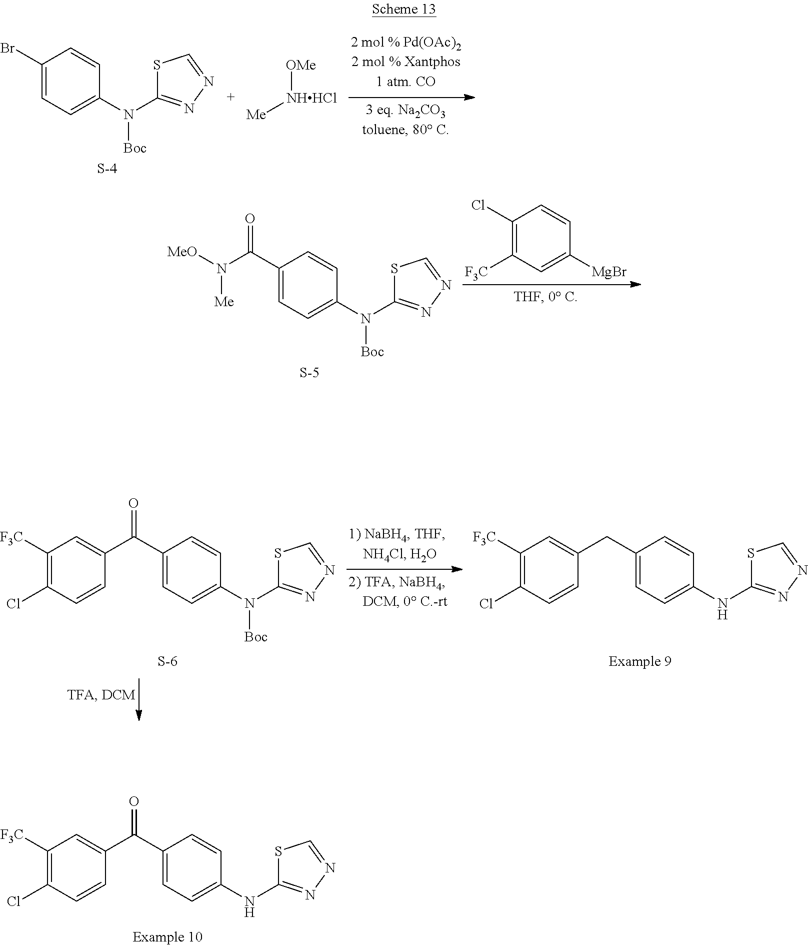

Referring to Scheme 13, Example 10 can be synthesized by converting Compound S-4 to the Weinreb amide via amino carbonylation using palladium acetate and Xantphos under atmospheric pressure of carbon monoxide. (Martinelli, J. R.; Watson, D. A.; Freckmann, D. M. M.; Barder, T. E.; Buchwald, S. L., J. Org. Chem. 2008, 73(18), 7102-7107.) The Weinreb amide can be treated with a Grignard reagent to provide the Compound S-6. N-Boc deprotection with TFA can provide Example 10. Alternatively, sequential reduction of the ketone with NaBH₄ and concomitant N-Boc deprotection can provide Example 9. (Banka, Anna et al, PCT Int. Appl., 2009/089454.)

### Example 11

### N-(4′-Chloro-3′-(trifluoromethyl)-[1,1′-biphenyl]-4-yl)-1,3,4-thiadiazol-2-amine

The title compound may be prepared as shown in Scheme 14, which provides an example of a synthetic route to a compound of Formula (I) in which X is absent.

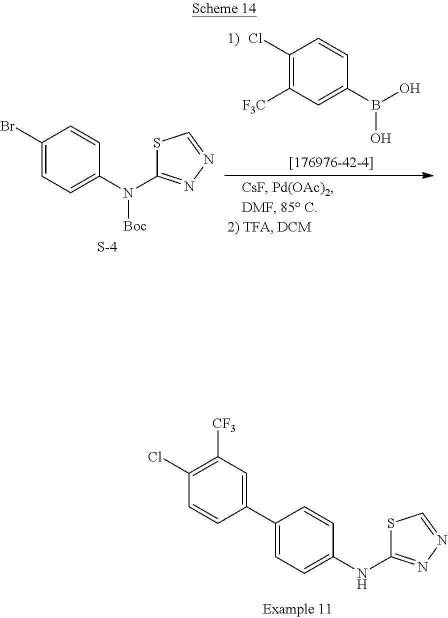

The synthesis may be accomplished via a Suzuki reaction. (Dorbec, Matthieu et al., Tetrahedron 2006, 62(50), 11766-11781.)

### Example 12

### N-(4-((4-Chloro-3-(trifluoromethyl)phenyl)sulfonyl)phenyl)-4H-1,2,4-triazol-3-amine and

### Example 13

### N-(4-((4-Chloro-3-(trifluoromethyl)phenyl)sulfonyl)phenyl)-1H-tetrazol-5-amine

Examples 12 and 13 may be prepared according to Scheme 15, which provides examples of synthetic routes to a compound of Formula (I) with triazole and tetrazole for Ring A.

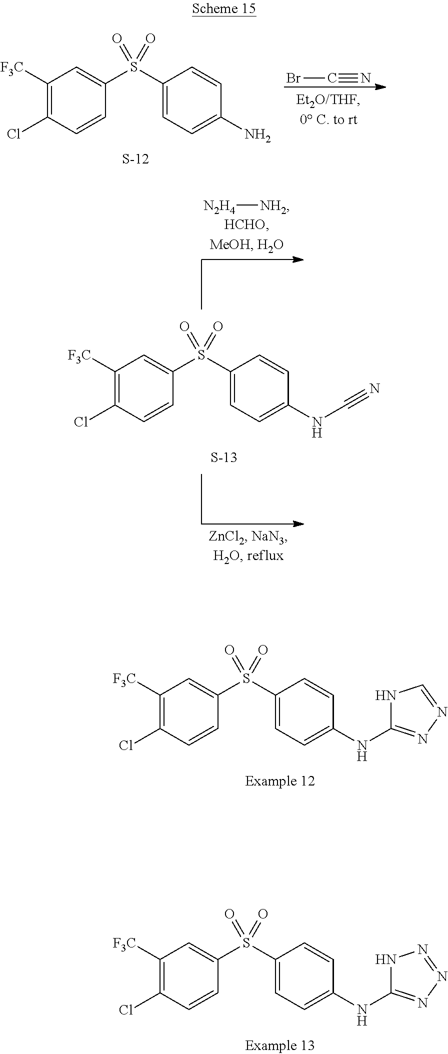

Referring to Scheme 15, the synthetic scheme of triazole Example 12 is shown. Compound S-12 can be treated with cyanogen bromide to make Compound S-13. Treatment with hydrazine and formaldehyde provides the triazole Example 12. (Deng, X., et. al., ACS Med. Chem. Lett. 2011, 2, 379-384.) Alternatively, treatment of Compound S-13 with sodium azide and zinc chloride in water under refluxing conditions provides the tetrazole Example 13. (By Habibi, D. et al., Tetrahedron 2010, 66(21), 3866-3870.)

### Example 14

### N-(4-((4-Chloro-3-(trifluoromethyl)phenyl)sulfonyl)phenyl)-1,3,4-oxadiazol-2-amine

Example 14 may be prepared as shown in Scheme 16, which provides an example of a synthetic route to a compound of Formula (I) with oxadiazole for Ring A.

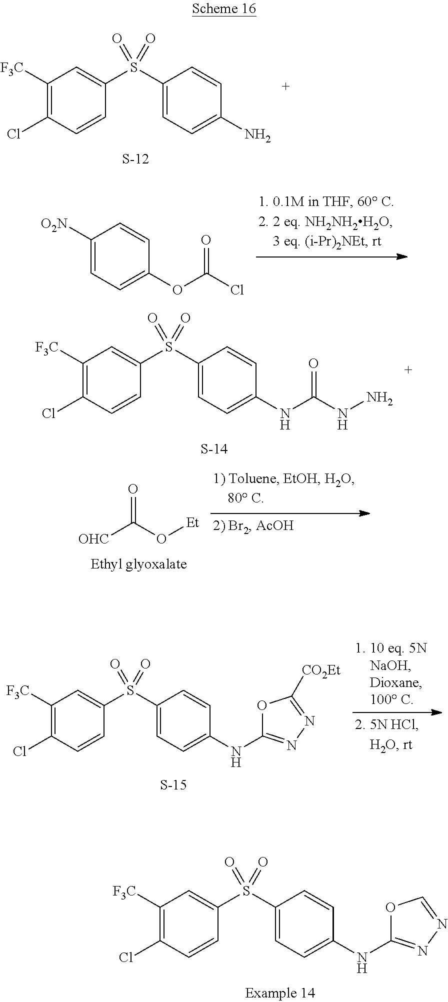

Referring to Scheme 16, oxadiazole Example 14 can be synthesized. (Berdini, Valerio et al., PCT Int. Appl., 2008078100, 3 Jul. 2008) Treatment of Compound S-12 with 4-nitrophenylchloroformate followed by addition of hydrazine hydrate with Huenig's base can give Compound S-14. Subsequent treatment with ethyl glyoxylate followed by cyclization with bromine in acetic acid can provide Compound 5-15. Decarboxylation of Compound 5-15 can give Example 14.

### Example 15

### N-(4-((4-Chloro-3-(trifluoromethyl)phenyl)sulfonyl)phenyl)-1H-imidazol-5-amine and

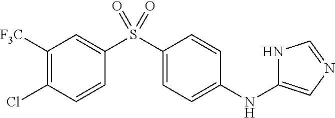 [SMILES: O=S(=O)(c1ccc(Nc2cnc[nH]2)cc1)c1ccc(Cl)c(C(F)(F)F)c1]

### Example 16

### N-(4-((4-Chloro-3-(trifluoromethyl)phenyl)sulfonyl)phenyl)-1H-pyrrol-2-amine

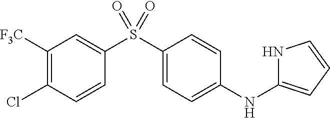 [SMILES: O=S(=O)(c1ccc(Nc2ccc[nH]2)cc1)c1ccc(Cl)c(C(F)(F)F)c1]

The title compounds may be prepared using methods analogous to those described in the general schemes and Examples.

### Example 17

### N-(6-((4-Chloro-3-(trifluoromethyl)phenyl)sulfonyl)pyridin-3-yl)-1,3,4-thiadiazol-2-amine

### Example 18

### N-(2-((4-Chloro-3-(trifluoromethyl)phenyl)sulfonyl)pyrimidin-5-yl)-1,3,4-thiadiazol-2-amine; and

### Example 19

### N-(5-((4-Chloro-3-(trifluoromethyl)phenyl)sulfonyl)pyrazin-2-yl)-1,3,4-thiadiazol-2-amine

The compounds of Examples 17-19 may be prepared according to Schemes 17 and 18, which provide examples of synthetic routes to a compound of Formula (I) with pyridines, pyrimidines, pyrazines, and triazines for the central aromatic ring.

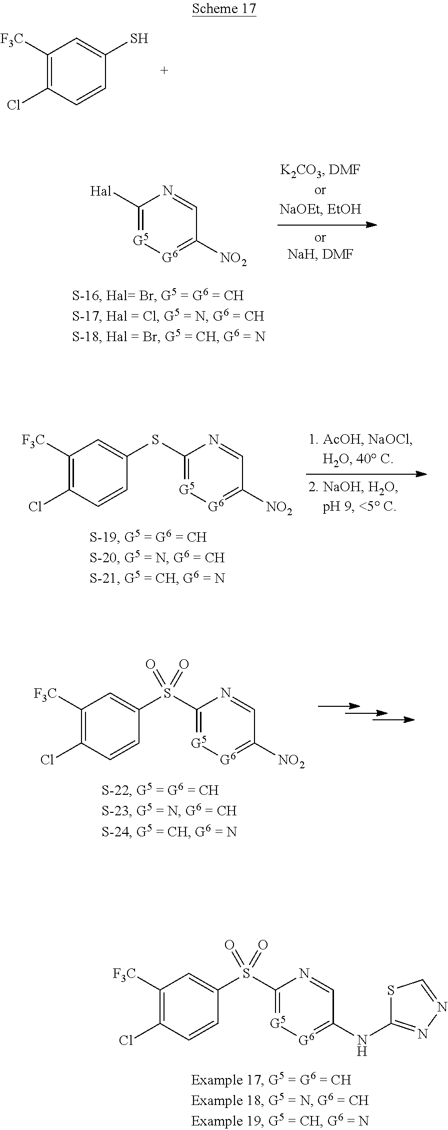

Referring to Scheme 17, SN_{Ar} reaction of Compounds S-16, S-17 or S-18 with 3-trifluoromethyl-4-chloro-thiophenol can provide Compound S-19 to S-21. The choice of base for the SN_{Ar} reaction depends on the starting heterocycle. For the Compound S-16, potassium carbonate can be used as a base. (Trankle, W. G.; Kopach, M. E., Org. Process Res. Dev., 2007, 11 (5), 913-917.) For Compound S-17, sodium ethoxide can be used for the SN_{Ar} (Ram, S. et al., J. Het. Chem., 1989, 26 (4), 1053-1059.), while for Compound S-18, sodium hydride could be used. (Charrier, Jean-Damien et al., PCT Int. Appl., 2011/143419). Oxidation of Compounds S-19 to S-21 can be achieved with hydrogen peroxide as described for the synthesis of Example 1, or with sodium hypochlorite as described by Trankle, supra. From Compounds S-19 to S-21, the syntheses of the compounds are achieved using methods similar to that described for the synthesis of Example 1.

An alternate synthesis of Example 19 is shown below in Scheme 18. (Wang, Tao et al., U.S. Patent Appl. Publ. No. 2004/0110785.)

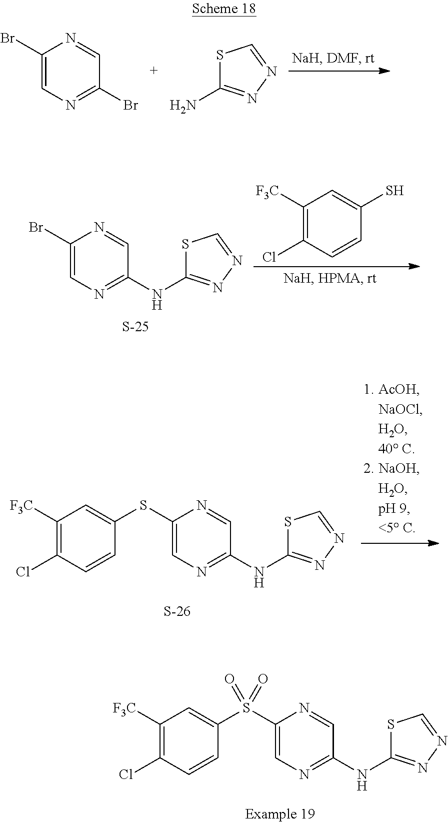

### Example 20

### N-(3-((4-Chloro-3-(trifluoromethyl)phenyl)sulfonyl)-1,2,4-triazin-6-yl)-1,3,4-thiadiazol-2-amine and

### Example 21

### N-(6-((4-Chloro-3-(trifluoromethyl)phenyl)sulfonyl)-1,2,4,5-tetrazin-3-yl)-1,3,4-thiadiazol-2-amine

Examples 20 and 21 may be prepared as shown in Scheme 19, which provides an example of the synthesis of compounds of Formula (I) in which the central aromatic ring is a triazine or tetrazine.

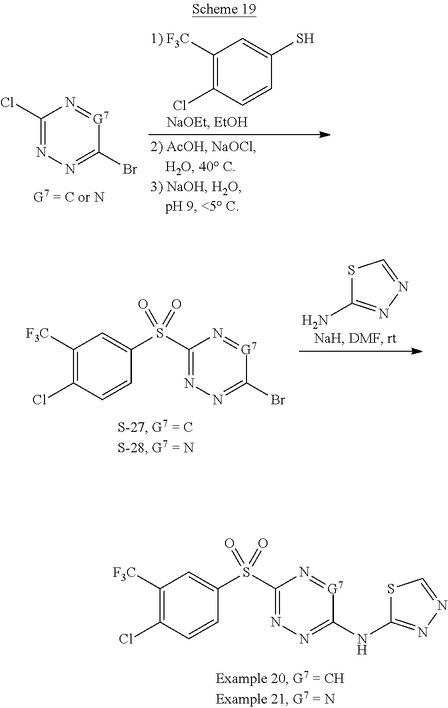

### Example 22

### N-(5-((4-Chlorophenyl)sulfonyl)pyrazin-2-yl)-1,3,4-thiadiazol-2-amine

### Example 23

### N-(5-((4-(Trifluoromethyl)phenyl)sulfonyl)pyrazin-2-yl)-1,3,4-thiadiazol-2-amine

### Example 24

### N-(5-((4-(Trifluoromethoxy)phenyl)sulfonyl)pyrazin-2-yl)-1,3,4-thiadiazol-2-amine; and

### Example 25

### N-(5-((4-Fluorophenyl)sulfonyl)pyrazin-2-yl)-1,3,4-thiadiazol-2-amine

Examples 22-25 may be prepared according to Scheme 20, which provides an example of the synthesis of compounds with various phenyl substituents R¹-R⁴ in Formula (I).

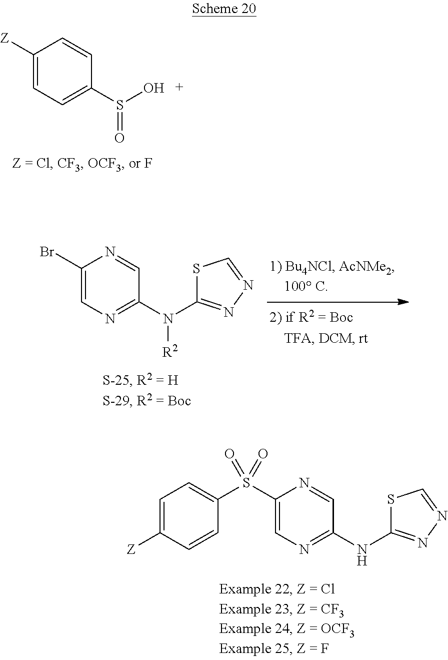

Referring to Scheme 20, the synthesis begins with the Compound S-25, described in the preceding examples, and involves reaction of a phenyl sulfonic acid with the bromo-pyrazine. (Maloney, Kevin M. et al., Org. Lett. 2011, 13(1), 102-105.)

### Example 26

### N-(5-((4-Chlorophenyl)sulfonyl)pyrazin-2-yl)-5-((dimethylamino)methyl)-1,3,4-thiadiazol-2-amine

### Example 27

### 5-(Aziridin-1-ylmethyl)-N-(5-((4-chlorophenyl) sulfonyl)pyrazin-2-yl)-1,3,4-thiadiazol-2-amine

### Example 28

### N-(5-((4-Chlorophenyl) sulfonyl)pyrazin-2-yl)-5-(piperidin-1-ylmethyl)-1,3,4-thiadiazol-2-amine

### Example 29

### N-(5-((4-Chlorophenyl)sulfonyl)pyrazin-2-yl)-5-((4-methylpiperazin-1-yl)methyl)-1,3,4-thiadiazol-2-amine; and

### Example 30

### N-(5-((4-Chlorophenyl) sulfonyl)pyrazin-2-yl)-5-(morpholinomethyl)-1,3,4-thiadiazol-2-amine

Examples 26-30 may be prepared as shown in Scheme 21, which provides an example of the synthesis of compounds of Formula (I) in which n is not zero.

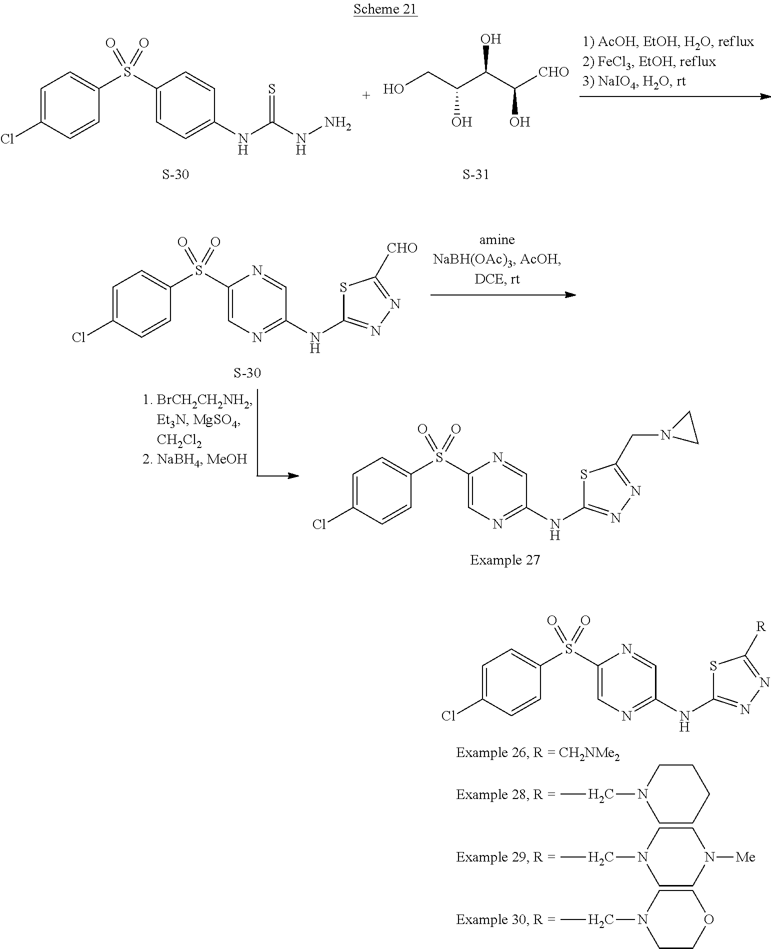

Referring to Scheme 21, Compound S-30 can be synthesized as described in the synthesis for Example 1. Condensation of Compound S-30 with D-arabinose could give the thiosemicarbazone, which can undergo oxidative cyclization and cleavage of the sugar chain with ferric chloride. Periodate oxidation could then give Compound S-32. (Shaban, M. A. E. et al., Pharmazie 2003, 58(6), 367-371.) With Compound S-32 in hand, the aldehyde can be treated with various amines under reductive amination conditions to provide Examples 26, 28, 29, and 30. The aziridine Example 27 can be synthesized from Compound S-32 in a stepwise manner, by first making the haloalkylimine and then reduction and cyclization with NaBH₄ in methanol. (De Kimpe, N.; De, Smaele, Tetrahedron 1994, 35(43), 8023-8026.)

### Example 31

### 2-(5-((5-((4-Chlorophenyl) sulfonyl)pyrazin-2-yl)amino)-1,3,4-thiadiazol-2-yl)ethanol

Example 31 may be prepared according to Scheme 22.

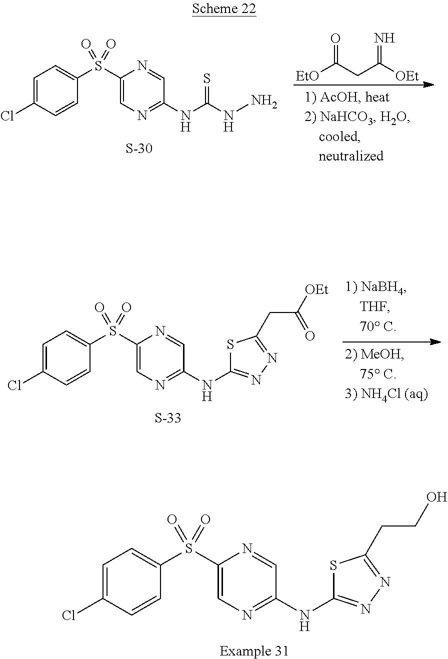

Referring to Scheme 22, Compound S-30 can be heated with 3-ethoxy-3-iminopropionate hydrochloride in glacial acetic acid to provide Compound S-33. (Ahad, Ali Md. et al., Bioorg. Med. Chem., 2011, 19(6), 2046-2054.) Reduction of the ester with sodium borohydride provides Example 31. (Boechat, N. et al. Syn. Comm. 2005, 35(24), 3187-3190.)

### Example 32

### 5-(Aminomethyl)-N-(5-((4-chlorophenyl)sulfonyl)pyrazin-2-yl)-1,3,4-thiadiazol-2-amine

### Example 33

### N-((5-((5-((4-Chlorophenyl)sulfonyl)pyrazin-2-yl)amino)-1,3,4-thiadiazol-2-yl)methyl)acetamide; and

### Example 34

### N-((5-((5-((4-Chlorophenyl)sulfonyl)pyrazin-2-yl)amino)-1,3,4-thiadiazol-2-yl)methyl)methanesulfonamide

Examples 32-34 may be synthesized as shown in Scheme 23.

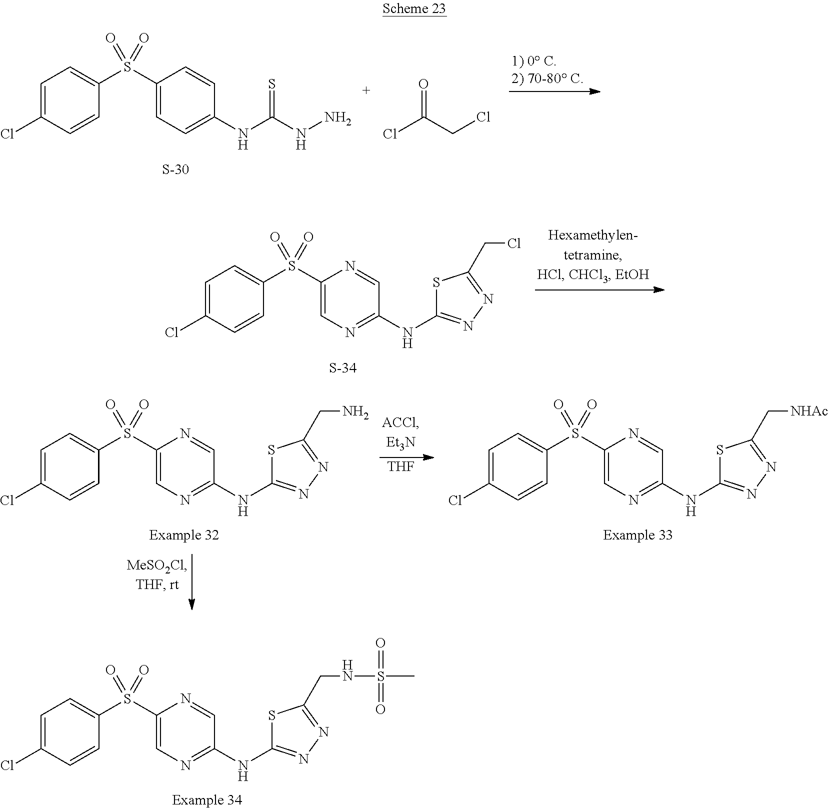

Referring to Scheme 23, treatment of Compound S-30 with chloroacetyl chloride with heating can provide Compound S-34. A Delepine reaction with hexamethylentetramine produced the aminomethyl derivative Example 32. (Serban, Georgeta et al, Analele Universitatii din Oradea, Fascicula Chimie, 2009, 16, 91-95.) This product can be reacted with acetyl chloride to make Example 33 or with methanesulfonyl chloride to make Example 34.

Examples 35-41 may be prepared using methods analogous to those described for Example 1, starting with the appropriately substituted aniline reagent.

### Assay Example 1

### Inhibition of PI3K-AKT-mTor Pathway—Quantification of Phosphorylated AKT and mTOR

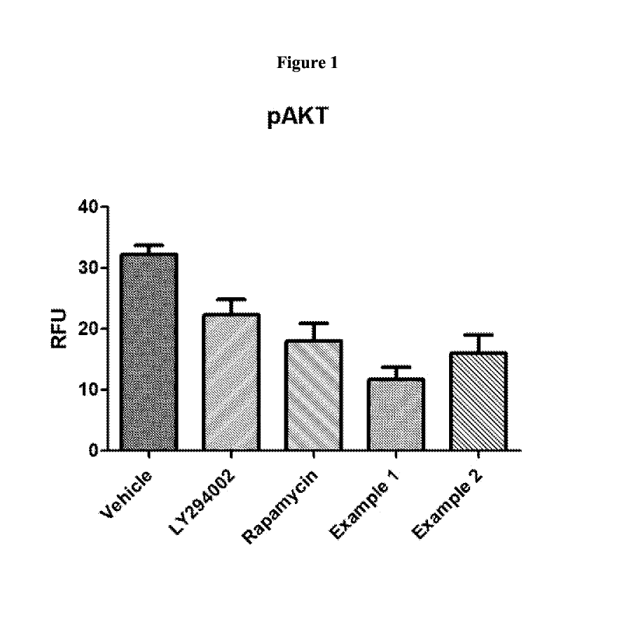

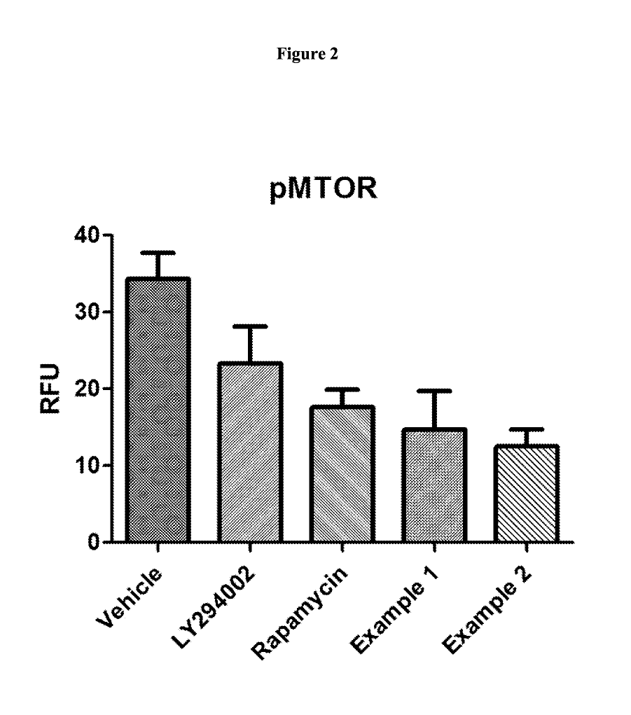

B103 neuroblastoma cells were plated at a density of 1×10⁶/10 cm dish and were maintained for 16 hours in Dulbecco's Modified Eagle Medium (DMEM; Fisher Scientific) with 10% fetal bovine serum (FBS; Life Technologies) and 1% Penicillin-Streptomycin (Life Technologies). Cells were treated with test compound and incubated for 30 minutes. Cells were then lysed with cell lysis buffer (Cell Signaling) and the lysate collected. Detection and ELISA quantification of phosphorylated AKT and mTOR was accomplished using Phospho-AKT and Phospho-mTOR antibodies (Cell Signaling) following the manufacturer's protocols. Data for compounds tested in these assays are presented in FIGS. 1 and 2. LY294002 and rapamycin are reference compounds known to modulate the PI3K-AKT-MTOR pathway.

### Assay Example 2

### Determining the Effects of Test Compounds on Autophagy by Measuring GFP-LC3

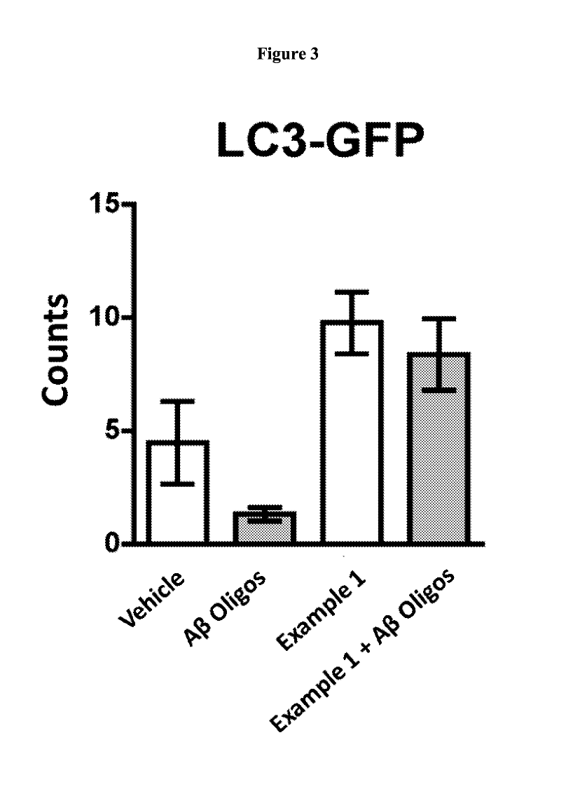

B103 neuroblastoma cells were maintained in DMEM as described in Assay Example 1 and infected with lentivirus expressing LC3 (microtubule associated protein 1 light chain 3) tagged with GFP (green fluorescent protein) at a MOI of 40. Increased LC3 incorporation into autophagosomes is a marker of increased autophagy. Forty-eight hours post infection, B103 cells were plated onto poly-L-lysine treated coverslips and treated with test compound for 16 hours. The cells were then fixed with 4% paraformaldehyde. Coverslips were digitally imaged and the expression of LC3-GFP in autophagosomes was quantified by counting the fluorescent puncta in cells. Data for compounds tested in this assay are presented in FIG. 3.

### Assay Example 3

### Determining the Effect of Test Compounds on the Clearance of Oligomeric Amyloid Beta

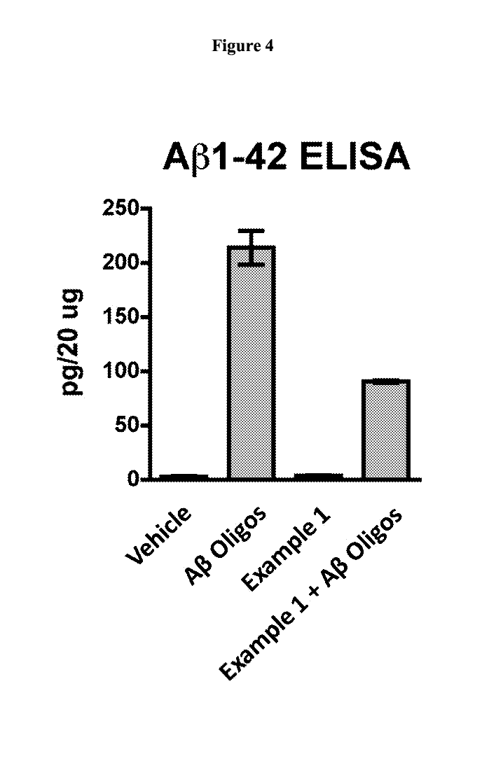

SH-SY5Y human neuroblastoma cells that express amyloid precursor protein (APP) were maintained in DMEM/F-12 (Life Technologies) with 10% FBS (Life Technologies), 1% Penicillin-Streptomycin-Glutamine (Life Technologies), 0.1% non-essential amino acids (Life Technologies) and 18 μM sodium bicarbonate. Gentamicin (Life Technologies) treatment induces APP expression by these cells. SH-SY5Y-APP cells were plated at a density of 1×10⁶/10 cm dish and treated with test compounds for 16 hours. The cells were then lysed with PDGF buffer (HEPES 1.0 mM, Benzamidine 5.0 mM, 2-Mercaptoethanol 2.0 mM, EDTA 3.0 mM, Magnesium Sulfate 0.5 mM, Sodium Azide 0.05% pH to 8.8) and collected. Protein concentration of the cell lysates was determined using the BCA protein assay according to the manufacturer's protocol (Thermo Scientific Pierce). The amount of amyloid beta retained in the cells was then measured using an ELISA assay specific for amyloid beta following the manufacturer's protocol (Life Technologies). Data for compounds tested in this assay are presented in FIG. 4.

1. A compound of Formula I: 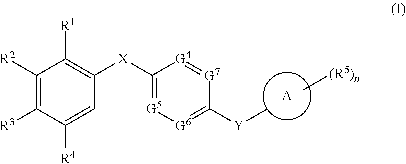
wherein
R¹, R², R³, and R⁴ are each independently hydrogen, hydroxy, halogen, C₁₋₄ alkyl, substituted C₁₋₄ alkyl, C₁₋₄ alkoxy, substituted C₁₋₄ alkoxy, —CN, —COR^{x}, —CO₂R^{x}, —SO₂R^{x}, or —NR^{x}R^{y};
wherein R^{x} and R^{y} are each independently H or optionally substituted C₁₋₄alkyl, or R^{x} and R^{y} taken together with the nitrogen to which they are attached form an optionally substituted monocyclic heterocycloalkyl ring;
X is absent, or is C₁₋₆ alkylene, wherein one carbon unit of said alkylene is optionally replaced with —O—, —S—, —SO—, —NR^{a}—, —SO₂—, or —CO—;
wherein R^{a} is hydrogen or C₁₋₄ alkyl;
G⁴, G⁵, G⁶, and G⁷ are each independently CR¹⁰ or N;
wherein each R¹⁰ is independently hydrogen, hydroxy, halogen, C₁₋₄ alkyl, C₁₋₄ haloalkyl, C₁₋₄ alkoxy, or C₁₋₄ haloalkoxy;
Y is absent, or is C₁₋₆ alkylene, wherein one carbon unit of said alkylene is optionally replaced with —O—, —S—, —NH—, —SO—, —SO₂—, —CO—, —CO₂—, —CONH—, —NHCO—, —NHSO₂—, or —SO₂NH—;
Ring A is a 5-membered heteroaryl ring;
each R⁵ is independently C₁₋₆ alkyl, substituted C₁₋₆ alkyl, C₁₋₆ alkoxy, substituted C₁₋₆ alkoxy, C₃₋₈ cycloalkyl, substituted C₃₋₈ cycloalkyl, C₃₋₈ cycloalkoxy, substituted C₃₋₈ cycloalkoxy, hydroxyl, halogen, —NR^{m}Rⁿ, or cyano;
wherein R^{m} and Rⁿ are each independently H or C₁₋₄alkyl; and
n is a number from zero to three;
or a pharmaceutically acceptable salt thereof.

2. The compound of claim 1, which is a compound of Formula (II): 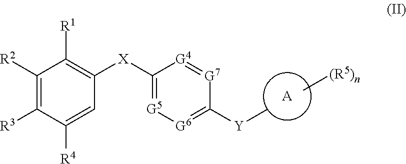
wherein
R¹, R², R³, and R⁴ are each independently hydrogen, hydroxy, halogen, C₁₋₄ alkyl, or C₁₋₄ alkoxy, wherein each alkyl or alkoxy is unsubstituted or substituted with one or more substituents independently selected from hydroxy, halogen, amino, cyano, and nitro;
X is absent, or is C₁₋₆ alkylene, wherein one carbon unit of said alkylene is optionally replaced with —O—, —S—, —SO—, —NR^{a}—, —SO₂—, or —CO—;
wherein R^{a} is hydrogen or C₁₋₄ alkyl;
G⁴, G⁵, G⁶, and G⁷ are each independently CR¹⁰ or N;
wherein each R¹⁰ is independently hydrogen, hydroxy, halogen, C₁₋₄ alkyl, C₁₋₄ haloalkyl, C₁₋₄ alkoxy, or C₁₋₄haloalkoxy;
Y is absent, or is C₁₋₆ alkylene, wherein one carbon unit of said alkylene is optionally replaced with —O—, —S—, —NH—, —SO—, —SO₂—, —CO—, —CO₂—, —CONH—, —NHCO—, —NHSO₂—, or —SO₂NH—;
Ring A is a 5-membered heteroaryl ring;
each R⁵ is independently C₁₋₆ alkyl, C₁₋₆ alkoxy, C₃₋₈ cycloalkyl, C₃₋₈ cycloalkoxy, hydroxyl, halogen, —NR^{m}Rⁿ, or cyano;
wherein R^{m} and Rⁿ are each independently H or C₁₋₄alkyl; and
each alkyl, alkoxy, cycloalkyl, or cycloalkoxy is unsubstituted or substituted with hydroxyl, halogen, —NR^{b}R^{c}, monocyclic heterocycloalkyl, or poly(alkylene glycol);
wherein said monocyclic heterocycloalkyl is unsubstituted or substituted with C₁₋₄alkyl, —SO₂C₁₋₄alkyl, —COC₁₋₄alkyl, or —CO₂C₁₋₄alkyl;
wherein R^{b} and R^{c} are each independently hydrogen, —C₁₋₄alkyl, —COC₁₋₄alkyl, —SO₂C₁₋₄alkyl, or —CO₂C₁₋₄alkyl;
wherein each alkyl is unsubstituted or substituted with hydroxyl, C₁₋₄alkoxy, halogen, or —SO₂C₁₋₄alkyl;
or R^{b} and R^{c} taken together with the nitrogen to which they are attached form a monocyclic heterocycloalkyl,
wherein the monocyclic heterocycloalkyl is unsubstituted or substituted with C₁₋₄alkyl, —SO₂C₁₋₄alkyl, —COC₁₋₄alkyl, or —CO₂C₁₋₄alkyl; and
n is a number from zero to three;
or a pharmaceutically acceptable salt thereof.

3. The compound of claim 1, which is a compound of Formula (III): 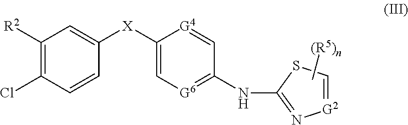
wherein
R² is H or —CF₃;
X is —SO₂—, —O—, —NH—, or —CO—;
G², G⁴, and G⁶ are each independently CH or N;
R⁵ is C₁₋₄alkyl optionally substituted with —NR^{b}R^{c};
wherein R^{b} and R^{c} are each independently H or C₁₋₄alkyl; or R^{b} and R^{c} taken together with the nitrogen to which they are attached form a monocyclic heterocycloalkyl ring, unsubstituted or substituted with C₁₋₄alkyl; and
n is zero or one;
or a pharmaceutically acceptable salt thereof.

4. The compound of claim 3, wherein X is —SO₂—.

5. The compound of claim 3, wherein G⁴ and G⁶ are each CH.

6. The compound of claim 3, wherein n is zero.

7. A compound selected from the group consisting of:
N-(4-((4-chloro-3-(trifluoromethyl)phenyl)sulfonyl)phenyl)-1,3,4-thiadiazol-2-amine;
N-(4-((4-chloro-3-(trifluoromethyl)phenyl)sulfonyl)phenyl) thiazol-2-amine;
N-(5-((4-chlorophenyl)sulfonyl)pyrazin-2-yl)-5-((4-(methylsulfonyl)piperazin-1-yl)methyl)-1,3,4-thiadiazol-2-amine;
N-(4-(4-chlorophenoxyl)phenyl)-1,3,4-thiadiazol-2-amine;
N¹-(4-chlorophenyl)-N⁴-(1,3,4-thiadiazol-2-yl)benzene-1,4-diamine;
(4-((1,3,4-thiadiazol-2-yl)amino)phenyl)(4-chlorophenyl)methanone;
N-(4-(4-chloro-3-(trifluoromethyl)phenoxy)phenyl)-1,3,4-thiadiazol-2-amine;
N¹-(4-chloro-3-(trifluoromethyl)phenyl)-N⁴-(1,3,4-thiadiazol-2-yl)benzene-1,4-diamine;
N-(4-(4-chloro-3-(trifluoromethyl)benzyl)phenyl)-1,3,4-thiadiazol-2-amine;
(4-((1,3,4-thiadiazol-2-yl)amino)phenyl)(4-chloro-3-(trifluoromethyl)phenyl)methanone;
N-(4′-chloro-3′-(trifluoromethyl)-[1,1′-biphenyl]-4-yl)-1,3,4-thiadiazol-2-amine;
N-(4-((4-chloro-3-(trifluoromethyl)phenyl)sulfonyl)phenyl)-4H-1,2,4-triazol-3-amine;
N-(4-((4-chloro-3-(trifluoromethyl)phenyl)sulfonyl)phenyl)-1H-tetrazol-5-amine;
N-(4-((4-chloro-3-(trifluoromethyl)phenyl)sulfonyl)phenyl)-1,3,4-oxadiazol-2-amine;
N-(4-((4-chloro-3-(trifluoromethyl)phenyl)sulfonyl)phenyl)-1H-imidazol-5-amine;
N-(4-((4-chloro-3-(trifluoromethyl)phenyl)sulfonyl)phenyl)-1H-pyrrol-2-amine;
N-(6-((4-chloro-3-(trifluoromethyl)phenyl)sulfonyl)pyridin-3-yl)-1,3,4-thiadiazol-2-amine;
N-(2-((4-chloro-3-(trifluoromethyl)phenyl)sulfonyl)pyrimidin-5-yl)-1,3,4-thiadiazol-2-amine;
N-(5-((4-chloro-3-(trifluoromethyl)phenyl)sulfonyl)pyrazin-2-yl)-1,3,4-thiadiazol-2-amine;
N-(3-((4-chloro-3-(trifluoromethyl)phenyl)sulfonyl)-1,2,4-triazin-6-yl)-1,3,4-thiadiazol-2-amine;
N-(6-((4-chloro-3-(trifluoromethyl)phenyl)sulfonyl)-1,2,4,5-tetrazin-3-yl)-1,3,4-thiadiazol-2-amine;
N-(5-((4-chlorophenyl)sulfonyl)pyrazin-2-yl)-1,3,4-thiadiazol-2-amine;
N-(5-((4-(trifluoromethyl)phenyl)sulfonyl)pyrazin-2-yl)-1,3,4-thiadiazol-2-amine;
N-(5-((4-(trifluoromethoxy)phenyl)sulfonyl)pyrazin-2-yl)-1,3,4-thiadiazol-2-amine;
N-(5-((4-fluorophenyl)sulfonyl)pyrazin-2-yl)-1,3,4-thiadiazol-2-amine;
N-(5-((4-chlorophenyl)sulfonyl)pyrazin-2-yl)-5-((dimethylamino)methyl)-1,3,4-thiadiazol-2-amine;
5-(aziridin-1-ylmethyl)-N-(5-((4-chlorophenyl)sulfonyl)pyrazin-2-yl)-1,3,4-thiadiazol-2-amine;
N-(5-((4-chlorophenyl)sulfonyl)pyrazin-2-yl)-5-(piperidin-1-ylmethyl)-1,3,4-thiadiazol-2-amine;
N-(5-((4-chlorophenyl)sulfonyl)pyrazin-2-yl)-5-((4-methylpiperazin-1-yl)methyl)-1,3,4-thiadiazol-2-amine;
N-(5-((4-chlorophenyl)sulfonyl)pyrazin-2-yl)-5-(morpholinomethyl)-1,3,4-thiadiazol-2-amine;
2-(5-((5-((4-chlorophenyl)sulfonyl)pyrazin-2-yl)amino)-1,3,4-thiadiazol-2-yl)ethanol;
5-(aminomethyl)-N-(5-((4-chlorophenyl)sulfonyl)pyrazin-2-yl)-1,3,4-thiadiazol-2-amine;
N-((5-((5-((4-chlorophenyl)sulfonyl)pyrazin-2-yl)amino)-1,3,4-thiadiazol-2-yl)methyl)acetamide;
N-((5-((5-((4-chlorophenyl)sulfonyl)pyrazin-2-yl)amino)-1,3,4-thiadiazol-2-yl)methyl)methanesulfonamide;
N-(4-((4-chloro-3-(trifluoromethyl)phenyl)sulfonyl)-3-fluorophenyl)-1,3,4-thiadiazol-2-amine;
N-(4-((3-(trifluoromethyl)phenyl)sulfonyl)phenyl)-1,3,4-thiadiazol-2-amine;
N-(2-chloro-4-((3-(trifluoromethyl)phenyl)sulfonyl)phenyl)-1,3,4-thiadiazol-2-amine;
5-bromo-N-(4-((3-(trifluoromethyl)phenyl)sulfonyl)phenyl)-1,3,4-thiadiazol-2-amine;
N-(4-((3-(trifluoromethoxy)phenyl)sulfonyl)phenyl)-1,3,4-thiadiazol-2-amine;
N-(4-((4-fluorophenyl)sulfonyl)phenyl)-1,3,4-thiadiazol-2-amine; and
N-(4-((2-chloro-4-fluorophenyl)sulfonyl)phenyl)-1,3,4-thiadiazol-2-amine;
and pharmaceutically acceptable salts thereof.

8. A pharmaceutical composition comprising (a) at least one compound of Formula (I) in claim 1, or a pharmaceutically acceptable salt thereof, and (b) a pharmaceutically acceptable excipient.

9. A method of treating a disease or medical condition associated with autophagy or the PI3K-AKT-MTOR pathway, comprising administering to a subject in need of such treatment an effective amount of at least one compound of Formula I as in claim 1, or a pharmaceutically acceptable salt thereof.

10. The method of claim 9, wherein the disease or medical condition is Alzheimer's Disease, Parkinson's Disease, fronto-temporal dementia, dementia with Lewy Bodies, PD dementia, multiple system atrophy, Huntington's disease, Amyotrophic lateral sclerosis, cancer, infection, Crohn's disease, heart disease, and aging.
